(19) United States
(12) Patent Application Publication
(10) Pub. No.: US 2008/0317858 A1
(43) Pub. Date: Dec. 25, 2008

(54) FORMULATIONS OF N-(2-ACETYL-4,6-DIMETHYLPHENYL)-3--2-THIOPHENECARBOXAMIDE

(76) Inventors: Jinling Chen, Houston, TX (US); Lian Rajewski, Lawrence, KS (US); Aaron Schoeneman, Lee's Summit, MO (US); Andrew M. Trammel, Olathae, KS (US); Kent Amsberry, Fishers, IN (US)

Correspondence Address: JONES DAY, 222 EAST 41ST ST, NEW YORK, NY, 10017, US

(21) Appl. No.: 12/145,452

(22) Filed: Jun. 24, 2008

Related U.S. Application Data
(60) Provisional application No. 60/937,215, filed on Jun. 25, 2007.

(51) Int. Cl. A61K 9/36; A61K 31/422; A61K 9/28; A61P 9/12; A61P 9/00

(52) U.S. Cl. 424/480; 514/380; 424/474

(57) ABSTRACT

Provided herein are intravenous and oral formulations of N-(2-acetyl-4,6-dimethylphenyl)-3-{[(3,4 dimethyl-5-isoxazolyl)amino]sulfonyl}-2-thiophenecarboxamide. Also provided are methods of making and using the formulations.

### PRIORITY CLAIM

This application claims priority to U.S. provisional application Ser. No. 60/937,215 filed Jun. 25, 2007 to Chen et al. The disclosure of the above referenced application is incorporated by reference in its entirety.

### FIELD

Provided herein are formulations of N-(2-acetyl-4,6-dimethylphenyl)-3-{[(3,4 dimethyl-5-isoxazolyl)amino]sulfonyl}-2-thiophenecarboxamide and methods for treating endothelin-mediated disorders using the same. In certain embodiments, provided herein are intravenous (IV) formulations. In certain embodiments, the formulations are oral tablets. Also provided are methods of making and using the formulations.

### BACKGROUND

N-(2-acetyl-4,6-dimethylphenyl)-3-{[(3,4 dimethyl-5-isoxazolyl)amino]sulfonyl}-2-thiophenecarboxamide modulates activity of the endothelin family of peptides and is useful for the treatment of endothelin-mediated disorders. Formulations containing N-(2-acetyl-4,6-dimethylphenyl)-3-{[(3,4 dimethyl-5-isoxazolyl)amino]sulfonyl}-2-thiophenecarboxamide may require storage for an extended period of time. Therefore, formulations of this compound that are stable are desired.

### SUMMARY

In one embodiment, provided herein are formulations of N-(2-acetyl-4,6-dimethylphenyl)-3-{[(3,4 dimethyl-5-isoxazolyl)amino]sulfonyl}-2-thiophenecarboxamide for IV administration and methods for treating endothelin mediated disorders using the same. In one embodiment, the IV formulations contain the compound and a buffer.

In one embodiment, provided herein are oral tablet formulations of N-(2-acetyl-4,6-dimethylphenyl)-3-{[(3,4 dimethyl-5-isoxazolyl)amino]sulfonyl}-2-thiophenecarboxamide and methods for treating endothelin mediated disorders using the same. The tablets contain one or more excipients selected from a buffer, a binding agent, a diluent, a lubricant and a coating agent.

Also provided are methods of making the formulations. Further provided are articles of manufacture containing packaging material, the formulation of N-(2-acetyl-4,6-dimethylphenyl)-3-{[(3,4 dimethyl-5-isoxazolyl)amino]sulfonyl}-2-thiophenecarboxamide and a label that indicates that the formulation is for treating an endothelin mediated disorder.

### BRIEF DESCRIPTION OF FIGURES

FIG. 1 provides a flow diagram for the manufacture of a 55 litre batch of N-(2-acetyl-4,6-dimethylphenyl)-3-{[(3,4 dimethyl-5-isoxazolyl)amino]sulfonyl}-2-thiophenecarboxamide sterile solution for injection (50 mg/ml).

FIG. 2 illustrates a representative manufacturing flow diagram for tablets with 1.0 mg N-(2-acetyl-4,6-dimethylphenyl)-3-{[(3,4 dimethyl-5-isoxazolyl)amino]sulfonyl}-2-thiophenecarboxamide.

FIG. 3 illustrates a representative manufacturing flow diagram for tablets with 10 or 50 mg N-(2-acetyl-4,6-dimethylphenyl)-3-{[(3,4 dimethyl-5-isoxazolyl)amino]sulfonyl}-2-thiophenecarboxamide.

### DETAILED DESCRIPTION

A. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein “drug” or “drug product” or “drug substance” refers to N-(2-acetyl-4,6-dimethylphenyl)-3-{[(3,4 dimethyl-5-isoxazolyl)amino]sulfonyl}-2-thiophenecarboxamide.

As used herein “subject” is an animal, such as a mammal, including human, such as a patient.

As used herein, “an endothelin-mediated disorder” is a condition that is caused by abnormal endothelin activity or one in which compounds that inhibit endothelin activity have therapeutic use. Such disorders include, but are not limited to hypertension, cardiovascular disease, asthma, inflammatory diseases, ophthalmologic disease, menstrual disorders, obstetric conditions, gastroenteric disease, renal failure, pulmonary hypertension, endotoxin shock, anaphylactic shock, or hemorrhagic shock.

As used herein, and unless otherwise specified, the terms “treat,” “treating” and “treatment” contemplate an action that occurs while a patient is suffering from the specified disease or disorder, which reduces the severity of the disease or disorder, or retards or slows the progression of the disease or disorder. Treatment also encompasses any pharmaceutical use of the compositions herein, such as use for treating pulmonary hypertension.

As used herein, amelioration of the symptoms of a particular disorder by administration of a particular pharmaceutical composition refers to any lessening, whether permanent or temporary, lasting or transient that can be attributed to or associated with administration of the composition.

As used herein, unless otherwise specified, the terms “prevent,” “preventing” and “prevention” contemplate an action that occurs before a patient begins to suffer from the specified disease or disorder, which inhibits or reduces the severity of the disease or disorder.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, and unless otherwise specified, the terms “therapeutically effective amount” and “effective amount” of a compound mean an amount sufficient to provide a therapeutic benefit in the treatment, prevent and/or management of a disease, to delay or minimize one or more symptoms associated with the disease or disorder to be treated. The terms “therapeutically effective amount” and “effective amount” can encompass an amount that improves overall therapy, reduces or avoids symptoms or causes of disease or disorder, or enhances the therapeutic efficacy of another therapeutic agent.

As used herein, and unless otherwise specified, the term “prophylactically effective amount” of a compound means an amount sufficient to prevent a disease or disorder, or one or more symptoms associated with the disease or disorder, or prevent its recurrence. The term “prophylactically effective amount” can encompass an amount that improves overall prophylaxis or enhances the prophylactic efficacy of another prophylactic agent.

The terms “co-administration” and “in combination with” include the administration of two therapeutic agents either simultaneously, concurrently or sequentially with no specific time limits. In one embodiment, both agents are present in the cell or in the patient's body at the same time or exert their biological or therapeutic effect at the same time. In one embodiment, the two therapeutic agents are in the same composition or unit dosage form. In another embodiment, the two therapeutic agents are in separate compositions or unit dosage forms. In some embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes. 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

B. N-(2-acetyl-4,6-dimethylphenyl)-3-{[(3,4 dimethyl-5-isoxazolyl)amino]sulfonyl}-2-thiophenecarboxamide

The structural formula for N-(2-acetyl-4,6-dimethylphenyl)-3-{[(3,4 dimethyl-5-isoxazolyl)amino]sulfonyl}-2-thiophenecarboxamide is as follows:

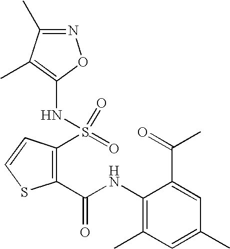

N-(2-acetyl-4,6-dimethylphenyl)-3-{[(3,4 dimethyl-5-isoxazolyl)amino]sulfonyl}-2-thiophenecarboxamide is an endothelin receptor antagonist that has oral bioavailability in several species, a long duration of action, and specificity for ETA receptors.

C. Exemplary Formulations

Provided herein are formulations of N-(2-acetyl-4,6-dimethylphenyl)-3-{[(3,4 dimethyl-5-isoxazolyl)amino]sulfonyl}-2-thiophenecarboxamide for IV and oral administration.

IV Formulations

In certain embodiments, provided herein are IV formulations containing N-(2-acetyl-4,6-dimethylphenyl)-3-{[(3,4 dimethyl-5-isoxazolyl)amino]sulfonyl}-2-thiophenecarboxamide and a buffer.

In certain embodiments, the IV formulations contain from about 0.5 mg/mL to about 60 mg/mL of N-(2-acetyl-4,6-dimethylphenyl)-3-{[(3,4 dimethyl-5-isoxazolyl)amino]sulfonyl}-2-thiophenecarboxamide. In certain embodiments, the IV formulations contain about 50 mg/mL of N-(2-acetyl-4,6-dimethylphenyl)-3-{[(3,4 dimethyl-5-isoxazolyl)amino]sulfonyl}-2-thiophenecarboxamide.

In certain embodiments, the IV formulations comprise sodium or potassium phosphate, or citrate buffer. In certain embodiments, the IV formulations provided herein comprise a phosphate buffer. In certain embodiments, the phosphate buffer is present in a concentration of about 10 mM, about 15 mM, about 20 mM, about 25 mM or about 30 mM. In certain embodiments, the phosphate buffer is present in a concentration of 20 mM.

In certain embodiments, the IV formulations have a pH in the range from 7-12 or 7-10. In one embodiment, the pH is 7, 7.5, 8, 8.5, 9, 9.5, 10, 10.5, 11, 11.5 or 12. In one embodiment, the pH is 7, 7.5, 8 or 8.5. In one embodiment, the pH is 8.

In certain embodiments, the phosphate buffer is present in a concentration of 20 mM, and the formulation has a pH of about 7.

The IV formulations provided herein are prepared by methods known to one of skill in the art. In certain embodiments, N-(2-acetyl-4,6-dimethylphenyl)-3-{[(3,4 dimethyl-5-isoxazolyl)amino]sulfonyl}-2-thiophenecarboxamide is solubilized using excess base equivalents of NaOH thus forming the sodium salt in situ. In one embodiment, about 1-2 equivalents of NaOH are used to solubilize the compound. In one embodiment, about 1.1-1.3 equivalents of NaOH are used to solubilize the compound. In one embodiment, about 1, 1.1, 1.2, 1.3, 1.4, 1.5, 1.6, 1.7, 1.8, 1.9 or 2 equivalents of NaOH are used. In one embodiment, 1.1 equivalent of NaOH is used.

In one embodiment, solubilization of the compound in the solvent is achieved with heat and high mixing rates. In one embodiment, the solubilization is achieved by heating the compound-solvent mixture from about 35° C. up to about 65° C. In one embodiment, the mixture is heated up to about 40° C., 42° C., 44° C., 46° C., 48° C., 50° C., 52° C., 54° C., 56° C., 58° C. or 600° C. In one embodiment, the mixture is heated up to about 50° C. In one embodiment, the solubilization is achieved at room temperature.

In one embodiment, the solubilization is carried out with mixing at about 100 rpm, 200 rpm, 250 rpm, 300 rpm, 350 rpm, 400 rpm or 500 rpm. In one embodiment, the solubilization is carried out with mixing at about 250 rpm.

In certain embodiments, the solubilization is carried out a higher pH, such as 12, 11, or 10 followed by lowering the pH to approximately pH 8 in the final formulation. In certain embodiments, the pH is lowered using a suitable acid, such as HCl, or a buffer. In certain embodiments, the compound is dissolved at about pH 12 and then the pH is lowered using HCl solution to approximately 8.

In another embodiment, in the process for preparation of the formulation a suitable buffer, such as a phosphate buffer is added to the formulation to bring the pH down to about 8. In other embodiment, N-(2-acetyl-4,6-dimethylphenyl)-3-{[(3,4 dimethyl-5-isoxazolyl)amino]sulfonyl}-2-thiophenecarboxamide is solubilized by 1.1 equivalents of NaOH at room temperature so that the compound concentration in NaOH solution is >50 mg/mL, the concentration is brought to 50 mg/mL by dilution with a phosphate buffer which also brings the pH down to approximately 8 in the same step. In one embodiment, 4 parts of N-(2-acetyl-4,6-dimethylphenyl)-3-{[(3,4 dimethyl-5-isoxazolyl)amino]sulfonyl}-2-thiophenecarboxamide/NaOH solution is combined with 1 part of 100 mM phosphate buffer. In one embodiment, the combination of the two solutions has a final buffer concentration of 20 mM. In one embodiment, the final pH of the solution is about 8 after combination. In one embodiment, the IV formulation contains N-(2-acetyl-4,6-dimethylphenyl)-3-{[(3,4 dimethyl-5-isoxazolyl)amino]sulfonyl}-2-thiophenecarboxamide concentration of 50 mg/mL and a phosphate buffer concentration of 20 mM and has a pH of about 8.

In one embodiment, a N-(2-acetyl-4,6-dimethylphenyl)-3-{[(3,4 dimethyl-5-isoxazolyl)amino]sulfonyl}-2-thiophenecarboxamide/NaOH solution is prepared by mixing 50 mg/mL compound with 1.1 equivalents of NaOH. Once N-(2-acetyl-4,6-dimethylphenyl)-3-{[(3,4 dimethyl-5-isoxazolyl)amino]sulfonyl}-2-thiophenecarboxamide is fully solubilized, sodium phosphate dibasic heptahydrate buffer is added to the solution. In the next step, sodium phosphate monobasic monohydrate buffer is added to the solution. The ratio of the two buffer salts is such that the final pH value is about 7-8. In one embodiment, the formulations provided herein contain 50 mg/mL N-(2-acetyl-4,6-dimethylphenyl)-3-{[(3,4 dimethyl-5-isoxazolyl)amino]sulfonyl}-2-thiophenecarboxamide in 20 or 50 mM phosphate buffer solution at pH 7 or 8.

In certain embodiments, the IV formulations provided herein are stable for about 1 day up to about 5 days at room temperature. In one embodiment, the solution is stable for about 1, 2, 3, 4 or 5 days. The stability of the formulation can be determined by measuring the potency and purity of the drug product. In one embodiment, the purity analysis is conducted by HPLC. In other embodiments, the IV formulations provided herein are stable for about 1 month, 2 months, 4 months, 6 months, 9 months, 12 months, 2 years, 3 years, 4 years or more at room temperature.

Tablet Formulations

In certain embodiments, provided herein are oral tablets containing N-(2-acetyl-4,6-dimethylphenyl)-3-{[(3,4 dimethyl-5-isoxazolyl)amino]sulfonyl}-2-thiophenecarboxamide. The tablets further contain intragranular components, granulating agents, extragranular components and coating components. In one embodiment, the oral tablet contains a pH adjustment agent, such as sodium hydroxide and a buffer, such as a phosphate buffer.

In certain embodiments, the intragranular components, include, but are not limited to a binding agent, such as hydroxypropyl cellulose; a diluent, such as lactose monohydrate, and microcrystalline cellulose; and a disintegrant, such as crospovidone.

In certain embodiments, the extragranular components, include, but are not limited to a disintegrant, such as crospovidone CL and a lubricant, such as magnesium stearate.

In certain embodiments, the amount of N-(2-acetyl-4,6-dimethylphenyl)-3-{[(3,4 dimethyl-5-isoxazolyl)amino]sulfonyl}-2-thiophenecarboxamide in the oral tablet is from about 0.5% to about 60% of the total weight of the composition. In certain embodiments, the amount of N-(2-acetyl-4,6-dimethylphenyl)-3-{[(3,4 dimethyl-5-isoxazolyl)amino]sulfonyl}-2-thiophenecarboxamide is from about 1% to about 60%, 1% to about 30% or 10% to about 25% of the total weight of the composition. In certain embodiments, the amount of N-(2-acetyl-4,6-dimethylphenyl)-3-{[(3,4 dimethyl-5-isoxazolyl)amino]sulfonyl}-2-thiophenecarboxamide is about 1%, 5%, 7%, 10%, 15%, 20%, 25%, 30%, 40% or 50% of the total weight of the composition. In certain embodiments, the amount of N-(2-acetyl-4,6-dimethylphenyl)-3-{[(3,4 dimethyl-5-isoxazolyl)amino]sulfonyl}-2-thiophenecarboxamide is about 1%, 10% or 50% of the total weight of the composition.

In certain embodiments, the oral tablet contains about 0.1 mg, 0.25 mg, 0.5 mg, 1 mg,2 mg,3 mg,4 mg,5 mg,6 mg,7 mg,9 mg, 10 mg, 12 mg, l5 mg,20 mg,25 mg,30 mg, 35 mg, 40 mg, 45 mg, 50 mg, 60 mg, 80 mg, 100 mg, 125 mg, 150 mg, 175 mg, 200 mg, 225 mg, 250 mg, 275 mg, or 300 mg of N-(2-acetyl-4,6-dimethylphenyl)-3-{[(3,4 dimethyl-5-isoxazolyl)amino]sulfonyl}-2-thiophenecarboxamide. In certain embodiments, the oral tablet contains about 1 mg, 10 mg or 50 mg of N-(2-acetyl-4,6-dimethylphenyl)-3-{[(3,4 dimethyl-5-isoxazolyl)amino]sulfonyl}-2-thiophenecarboxamide.

In certain embodiments, the tablets contain a combination of diluents, such as microcrystalline cellulose and lactose monohydrate. In certain embodiments, the amount of lactose monohydrate in the oral tablet is from about 10% to about 80% of the total weight of the composition. In certain embodiments, the amount of lactose monohydrate is from about 20%, 40%, 50%, 60%, 70% or 75%, of the total weight of the tablet. In certain embodiments, the amount of lactose monohydrate is about 20%, 50%, 55%, 60%, 65%, 66%, 67%, 67.5%, 68%, 68.2%, 68.4%, 68.6%, 68.8%, 68.87%, 68.9%, 68.95%, 69%, 69.5%, 70% or 72% of the total weight of the tablet. In certain embodiments, the amount of lactose monohydrate is about 68.87% of the total weight of the tablet. In certain embodiments, the amount of lactose monohydrate is about 20% or 60% of the total weight of the tablet.

In certain embodiments, the amount of lactose monohydrate 310 is from about 40 mg to about 80 mg, from about 50 mg to about 75 mg, from about 60 mg to about 70 mg. In certain embodiments, the amount of lactose monohydrate 310 is about 50 mg, 55 mg, 60 mg, 65 mg, 68 mg, 68.2 mg, 68.4 mg, 68.6 mg, 68.8 mg, 68.87 mg, 68.9 mg, 68.95 mg, 69 mg, 70 mg, 71 mg, 72 mg, 74 mg or 75 mg. In certain embodiments, the amount of lactose monohydrate 310 is about 68.87 mg. In certain embodiments, the amount of lactose monohydrate 310 is about 20 or 60 mg.

In certain embodiments, the amount of microcrystalline cellulose (Avicel PH 101) in the oral tablet is from about 5% to about 40% of the total weight of the tablet. In certain embodiments, the amount of microcrystalline cellulose (Avicel PH 101) is from about 10% to about 35%, from about 15% to about 30%, from about 15% to about 25% of the total weight of the tablet. In certain embodiments, the amount of microcrystalline cellulose (Avicel PH 101) is about 10%, 15%, 17%, 18%, 20%, 23%, 25%, 27%, 30% or 40% of the total weight of the tablet. In certain embodiments, the amount of microcrystalline cellulose (Avicel PH 101) is about 20% of the total weight of the tablet.

In certain embodiments, the amount of microcrystalline cellulose (Avicel PH 101) in the oral tablet is from about 5 mg to about 40 mg. In certain embodiments, the amount of microcrystalline cellulose (Avicel PH 101) is from about 10 mg to about 35 mg or about 15 mg to about 25 mg. In certain embodiments, the amount of microcrystalline cellulose (Avicel PH 101) is about 10 mg, 15 mg, 17 mg, 18 mg, 19 mg, 20 mg, 22 mg, 24 mg, 26 mg, 28 mg or 30 mg. In certain embodiments, the amount of microcrystalline cellulose (Avicel PH 101) in the oral tablet is about 20 mg.

In certain embodiments, the binding agent is hydroxypropyl cellulose (Klucel FXF). In certain embodiments, the amount of hydroxypropyl methylcellulose (Klucel EXF) in the tablet is from about 1% to about 10% of the total weight of the composition. In certain embodiments, the amount of hydroxypropyl methylcellulose (Klucel EXF) is from about 1% to about 8%, from about 2% to about 6% or from about 3% to about 5% of the total weight of the tablet. In certain embodiments, the amount of hydroxypropyl methylcellulose (Klucel EXF) is about 1%, 2%, 3%, 4%, 5%, 6%, 7%, 8%, 9% or 10% of the total weight of the tablet. In certain embodiments, the amount of hydroxypropyl methylcellulose (Klucel EXF) is about 4% of the total weight of the tablet.

In certain embodiments, the amount of hydroxypropyl methylcellulose (Klucel EXF) in the tablet is from about 1 mg to about 10 mg, about 2 mg to about 8 mg or about 3 mg to about 5 mg. In certain embodiments, the amount of hydroxypropyl methylcellulose (Klucel EXF) in the tablet is about 1 mg, 2 mg, 3 mg, 4 mg, 5 mg, 6 mg, 7 mg, 8 mg or about 10 mg. In certain embodiments, the amount of hydroxypropyl methylcellulose (Klucel EXF) in the tablet is about 4 mg.

In one embodiment, the tablets provided herein contain a pH adjustment agent and a buffering agent as granulating agents. In one embodiment, the pH adjustment agent is sodium hydroxide and the buffering agent is sodium phosphate monobasic. In certain embodiments, the tablet contains about 0.01 to about 1% sodium hydroxide by total weight of the tablet. In certain embodiments, the tablet contains about 0.01%, 0.03%, 0.05%, 0.07%, 0.09%, 0.1%, 0.15%, 0.2%, 0.5%, 0.7% or 1% sodium hydroxide by total weight of the tablet. In certain embodiments, the tablet contains about 0.001 to about 0.1% sodium phosphate monobasic by total weight of the tablet. In certain embodiments, the tablet contains about 0.001%, 0.005%, 0.01%, 0.02%, 0.03%, 0.04%, 0.05% or 0.1% sodium phosphate monobasic by total weight of the tablet. In certain embodiments, the tablet contains about 0.03% sodium phosphate monobasic by total weight of the tablet.

In certain embodiments, the tablet contains about 0.01 to about 1 mg sodium hydroxide. In certain embodiments, the tablet contains about 0.01 mg, 0.05 mg, 0.1 mg, 0.2 mg, 0.5 mg, 0.7 mg or 1 mg sodium hydroxide. In certain embodiments, the tablet contains about 0.1 mg sodium hydroxide.

In certain embodiments, the tablet contains about 0.001 to about 0.1 mg sodium phosphate monobasic by total weight of the tablet. In certain embodiments, the tablet contains about 0.001 mg, 0.005 mg, 0.01 mg, 0.02 mg, 0.03 mg, 0.04 mg, 0.05 mg or 0.1 mg sodium phosphate monobasic. In certain embodiments, the tablet contains about 0.03 mg sodium phosphate monobasic.

In certain embodiments, the tablets provided herein contain crospovidone CL and magnesium stearate as extragranular components. In certain embodiments, the crospovidone CL is present from about 0.5% up to about 5% by total weight of the tablet. In one embodiment. the crospovidone CL is present in about 0.5%, 1%, 2%, 2.5%, 3%, 3.5%, 4%, 4.5% or 5% by total weight of the tablet. In one embodiment, the crospovidone CL is present in about 1% or 4% of the total weight of the tablet. In certain embodiments, the crospovidone CL is present from about 0.0 mg up to about 5 mg. In one embodiment, the crospovidone CL is present in about 0.5 mg, 1 mg, 2 mg, 2.5 mg, 3 mg, 3.5 mg, 4 mg, 4.5 mg or 5 mg by total weight of the tablet. In one embodiment, the crospovidone CL is present in about 1 mg or 4 mg of the total weight of the tablet.

In certain embodiments, the magnesium stearate is present from about 0.1% up to about 5% by total weight of the tablet. In one embodiment, the magnesium stearate is present in about 0.1%, 0.3%, 0.5%, 0.7%, 1%, 1.5%, 2%, 2.5%, 3%, 4% or about 5% by total weight of the tablet. In one embodiment, the magnesium stearate is present in about 1% or 4% of the total weight of the tablet. In certain embodiments, the magnesium stearate is present from about 0.5 mg up to about 5 mg. In one embodiment, the magnesium stearate is present in about 0.5 mg, 1 mg, 2 mg, 2.5 mg, 3 mg, 3.5 mg, 4 mg, 4.5 mg or 5 mg by total weight of the tablet. In one embodiment, the magnesium stearate is present in about 1 mg or 4 mg of the total weight of the tablet.

In certain embodiments, the tablets are coated with a coating component. Suitable coating materials are known in the art. In certain embodiments, the coating provides taste masking. In one embodiment, the coating provides clinical blinding. In one embodiment, the coating component is Opadry Yellow 03F92230, referred to as Opadry Yellow. In one embodiment, Opadry Yellow is present from about 1% up to about 5%. In another embodiment, Opadry Yellow is present in about 1%, 2%, 3%, 4% or 5%. In another embodiment, Opadry Yellow is present in about 3%. In one embodiment, Opadry Yellow is present from about 1 mg up to about 5 mg by total weight of the tablet. In another embodiment, Opadry Yellow is present in about 1 mg, 2 mg, 3 mg, 4 mg or 5 mg. In another embodiment, Opadry Yellow is present in about 3 mg.

In certain embodiments, the tablet contains, as intragranular components, hydroxypropyl cellulose (Klucel EXF), lactose monohydrate 310, microcrystalline cellulose, cospovidone CL: as granulating agents. N-(2-acetyl-4,6-dimethylphenyl)-3-{[(3,4 dimethyl-5-isoxazolyl)amino]sulfonyl}-2-thiophenecarboxamide, sodium hydroxide, sodium phosphate monobasic; and as extragranular agents, crospovidone CL, and magnesium stearate. In one embodiment, the tablet further contains a coating of Opadry yellow.

In certain embodiments, the tablet contains, as intragranular components, about 4.0% hydroxypropyl cellulose (Klucel EXF), about 68.87% lactose monohydrate 310, about 20% microcrystalline cellulose, about 1% cospovidone CL; as granulating agents, about 1% N-(2-acetyl-4,6-dimethylphenyl)-3-{[(3,4 dimethyl-5-isoxazolyl)amino]sulfonyl}-2-thiophenecarboxamide, about 0.1% sodium hydroxide, about 0.1% sodium phosphate monobasic; and as extragranular agents, about 4% crospovidone CL, and about 4% magnesium stearate. In one embodiment, the tablet further contains a coating of Opadry yellow at about 3%.

In certain embodiments, the tablet contains, as intragranular components, about 4.0 mg hydroxypropyl cellulose (Klucel EXF), about 68.8 mg lactose monohydrate 310, about 20 mg microcrystalline cellulose, about 1 mg cospovidone CL; as granulating agents, about 1 mg N-(2-acetyl-4,6-dimethylphenyl)-3-{[(3,4 dimethyl-5-isoxazolyl)amino]sulfonyl}-2-thiophenecarboxamide, about 0.1 mg sodium hydroxide, about 0.1 mg sodium phosphate monobasic; and as extragranular agents, about 4 mg crospovidone CL, and about 4 mg magnesium stearate. In one embodiment, the tablet further contains a coating of Opadry yellow at about 3 mg.

In certain embodiments, the tablet contains, as intragranular components, about 10% milled N-(2-acetyl-4,6-dimethylphenyl)-3-{[(3,4 dimethyl-5-isoxazolyl)amino]sulfonyl}-2-thiophenecarboxamide, about 4.0% hydroxypropyl cellulose (Klucel EXF), about 60% lactose monohydrate 310, about 20% microcrystalline cellulose, about 1% cospovidone CL; and as extragranular agents, about 4% crospovidone CL and about 4% magnesium stearate. In one embodiment, the tablet further contains a coating of Opadry yellow at about 3%.

In certain embodiments, the tablet contains, as intragranular components, about 10 mg milled N-(2-acetyl-4,6-dimethylphenyl)-3-{[(3,4 dimethyl-5-isoxazolyl)amino]sulfonyl}-2-thiophenecarboxamide, about 4.0 mg hydroxypropyl cellulose (Klucel EXF), about 60 mg lactose monohydrate 310, about 20 mg microcrystalline cellulose, about 1% cospovidone CL; and as extragranular agents, about 4 mg crospovidone CL and about 4 mg magnesium stearate. In one embodiment, the tablet further contains a coating of Opadry yellow at about 3 mg.

In certain embodiments, the tablet contains, as intragranular components, about 50% milled N-(2-acetyl-4,6-dimethylphenyl)-3-{[(3,4 dimethyl-5-isoxazolyl)amino]sulfonyl}-2-thiophenecarboxamide, about 4.0% hydroxypropyl cellulose (Klucel EXF), about 20% lactose monohydrate 310, about 20% microcrystalline cellulose, about 1% cospovidone CL; and as extragranular agents, about 4% crospovidone CL and about 4% magnesium stearate. In one embodiment, the tablet further contains a coating of Opadry yellow at about 3%.

In certain embodiments, the tablet contains, as intragranular components, about 50 mg milled N-(2-acetyl-4,6-dimethylphenyl)-3-{[(3,4 dimethyl-5-isoxazolyl)amino]sulfonyl}-2-thiophenecarboxamide, about 4.0 mg hydroxypropyl cellulose (Klucel EXF), about 60 mg lactose monohydrate 310, about 20 mg microcrystalline cellulose, about 1% cospovidone CL; and as extragranular agents, about 4 mg crospovidone CL and about 4 mg magnesium stearate. In one embodiment, the tablet further contains a coating of Opadry yellow at about 3 mg.

Exemplary tablet compositions with 1 mg, 10 mg and 50 mg N-(2-acetyl-4,6-dimethylphenyl)-3-{[(3,4 dimethyl-5-isoxazolyl)amino]sulfonyl}-2-thiophenecarboxamide are provided in Tables I-III.

TABLE I
Composition of N-(2-acetyl-4,6-dimethylphenyl)-3-{[(3,4 dimethyl-5-
isoxazolyl)amino] sulfonyl}-2-thiophenecarboxamide
1.0 mg Coated Tablets

| | mg per | % |
|---|---|---|
| Component | Tablet | w/w |
| Intragranular Components | | |
| Hydroxypropyl Cellulose (Klucel EXF) | 4.00 | 4.00 |
| Lactose Monohydrate 310 | 68.87 | 68.87 |
| Microcrystalline Cellulose (Avicel | 20.00 | 20.00 |
| PH101) | | |
| Crospovidone CL (Kollidone CL) | 1.00 | 1.00 |
| Granulating Agents | | |
| Milled Drug Substance | 1.00 | 1.00 |
| Sodium Hydroxide | 0.10 | 0.10 |
| Sodium Phosphate Monobasic, Granular | 0.03 | 0.03 |
| AR | | |
| Purified Water¹ | | |
| Extragranular Components | | |
| Crospovidone CL (Kollidone CL) | 4.00 | 4.00 |
| Magnesium Stearate (Non-Bovine | 1.00 | 1.00 |
| #5712) | | |
| Total Core Tablet Weight | 100.0 | 100.0 |
| Coating Components | | |
| Opadry Yellow 03F92230 | 3.00 | 3.00 |
| Purified Water¹ | | |
| Total Coated Tablet Weight | 103.0 | |

¹In-process agent. It is removed during the process.

TABLE II
Composition of N-(2-acetyl-4,6-dimethylphenyl)-3-{[(3,4 dimethyl-5-
isoxazolyl)amino] sulfonyl}-2-thiophenecarboxamide
10 mg Coated Tablets

| | mg per | % |
|---|---|---|
| Component | Tablet | w/w |
| Intragranular Components | | |
| Milled Drug Substance | 10.00 | 10.00 |
| Hydroxypropyl Cellulose (Klucel EXF) | 4.00 | 4.00 |
| Lactose Monohydrate 310 | 60.00 | 60.00 |
| Microcrystalline Cellulose (Avicel PH101) | 20.00 | 20.00 |
| Crospovidone CL (Kollidone CL) | 1.00 | 1.00 |
| Granulating Agents | | |
| Purified Water¹ | | |
| Extragranular Components | | |
| Crospovidone CL (Kollidone CL) | 4.00 | 4.00 |
| Magnesium Stearate (Non-Bovine #5712) | 1.00 | 1.00 |
| Total Core Tablet Weight | 100.0 | 100.0 |
| Coating Components | | |
| Opadry Yellow 03F92230 | 3.00 | 3.00 |
| Purified Water¹ | | |
| Total Coated Tablet Weight | 103.0 | |

¹In-process agent. It is removed during the process.

TABLE III
Composition of N-(2-acetyl-4,6-dimethylphenyl)-3-{[(3,4 dimethyl-5-
isoxazolyl)amino] sulfonyl}-2-thiophenecarboxamide
50 mg Coated Tablets

| | mg per | % |
|---|---|---|
| Component | Tablet | w/w |
| Intragranular Components | | |
| Milled Drug Substance | 50.00 | 50.00 |
| Hydroxypropyl Cellulose (Klucel EXF) | 4.00 | 4.00 |
| Lactose Monohydrate 310 | 20.00 | 20.00 |
| Microcrystalline Cellulose (Avicel PH101) | 20.00 | 20.00 |
| Crospovidone CL (Kollidone CL) | 1.00 | 1.00 |
| Granulating Agents | | |
| Purified Water¹ | | |
| Extragranular Components | | |
| Crospovidone CL (Kollidone CL) | 4.00 | 4.00 |
| Magnesium Stearate (Non-Bovine #5712) | 1.00 | 1.00 |
| Total Core Tablet Weight | 100.0 | 100.0 |
| Coating Components | | |
| Opadry Yellow 03F92230 | 3.00 | 3.00 |
| Purified Water¹ | | |
| Total Coated Tablet Weight | 103.0 | |

¹In-process agent. It is removed during the process.

D. Dosages

In human therapeutics, the physician will determine the dosage regimen that is most appropriate according to a preventive or curative treatment and according to the age, weight, stage of the disease and other factors specific to the subject to be treated. In certain embodiments, dose rates of are from about 1 to about 350 mg per day for an adult, from about 1 to about 300 mg per day, from about 5 to about 250 mg per day, from about 5 to about 250 mg per day or from about 10 to 50 mg per day for an adult. Dose rates of from about 50 to about 300 mg per day are also contemplated herein. In certain embodiments, doses are about 5 mg, 10 mg, 15 mg, 20 mg, 25 mg, 30 mg, 35 mg, 40 mg, 45 mg, 50 mg, 60 mg, 70 mg, 80 mg, 100 mg, 125 mg, 150 mg, 175 mg or 200 mg per day per adult.

The amount of N-(2-acetyl-4,6-dimethylphenyl)-3-{[(3,4 dimethyl-5-isoxazolyl)amino]sulfonyl}-2-thiophenecarboxamide in the formulations provided herein which will be effective in the prevention or treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response. and the past medical history of the subject.

Exemplary doses of a formulation include milligram or microgram amounts of the active compound per kilogram of subject or sample weight (e.g., from about 1 micrograms per kilogram to about 3 milligrams per kilogram, from about 10 micrograms per kilogram to about 3milligrams per kilogram, from about 10 micrograms per kilogram to about 3 milligrams per kilogram, or from about 100 microgram per kilogram to about 2 milligrams per kilogram). In certain embodiments, the amount of N-(2-acetyl-4,6-dimethylphenyl)-3-{[(3,4 dimethyl-5-isoxazolyl)amino]sulfonyl}-2-thiophenecarboxamide administered is from about 0.01 to about 3 mg/kg for a subject in need thereof. In certain embodiments, the amount of N-(2-acetyl-4,6-dimethylphenyl)-3-{[(3,4 dimethyl-5-isoxazolyl)amino]sulfonyl}-2-thiophenecarboxamide administered is about 0.01, 0.05, 0.1, 0.2, 0.4, 0.8, 1.5, 2, 3 mg/kg of a subject. In certain embodiments, the administration of N-(2-acetyl-4,6-dimethylphenyl)-3-{[(3,4 dimethyl-5-isoxazolyl)amino]sulfonyl}-2-thiophenecarboxamide is by intravenous injection.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In another embodiment, the dosage of the formulation provided herein is administered to prevent, treat, manage, or ameliorate a disorder, or one or more symptoms thereof in a subject in a unit dose of from about 1 mg to 300 mg, 50 mg to 250 mg or 75 mg to 200 mg. In another embodiment, the dosage of the formulation provided herein is administered to prevent, treat, manage, or ameliorate a disorder, or one or more symptoms thereof in a subject in a unit dose of about 1 mg, 10 mg or 50 mg.

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

E. Methods of Preparation

N-(2-acetyl-4,6-dimethylphenyl)-3-{[(3,4 dimethyl-5-isoxazolyl)amino]sulfonyl}-2-thiophenecarboxamide can be prepared by methods known in the art. An exemplary methods for the preparation are described in Examples 1 and 2. (Also see, U.S. Pat. No. 6,686,382).

The IV and tablet formulations of N-(2-acetyl-4,6-dimethylphenyl)-3-{-[(3,4 dimethyl-5-isoxazolyl)amino]sulfonyl}-2-thiophenecarboxamide can be prepared by methods known in the art and as described herein. Exemplary processes for producing the IV and tablet formulations are described in Examples section.

F. Evaluation of the Activity

Standard physiological, pharmacological and biochemical procedures are available and are known to one of skill in the art (see, for example U.S. Pat. Nos. 6,432,994; 6,683,103; 6,686,382; 6,248,767; 6,852,745; 5,783,705; 5,962,490; 5,594,021; 5,571821; 5,591,761; 5,514,691. 5,352,800, 5,334,598, 5,352,659, 5,248,807, 5,240,910, 5,198,548, 5,187,195, 5,082,838, 6,953,780, 6,946,481, 6,852,745, 6,835,741, 6,673,824, 6,670,367 and 6,670,362) to test the efficacy of N-(2-acetyl-4,6-dimethylphenyl)-3-{[(3,4 dimethyl-5-isoxazolyl)amino]sulfonyl}-2-thiophenecarboxamide formulations in the methods provided herein.

G. Methods of Treating

Methods for the treatment of endothelin-mediated disorders by administering the lyophilized formulations provided herein. In certain embodiments, the disorder is selected from hypertension, cardiovascular disease, asthma, pulmonary hypertension, inflammatory diseases, ophthalmologic disease, menstrual disorders, obstetric conditions, wounds, gastroenteric disease, renal failure, immunosuppressant-mediated renal vasoconstriction, erythropoietin-mediated vasoconstriction, endotoxin shock, anaphylactic shock and hemorrhagic shock. In one embodiment, the disorder is pulmonary hypertension.

H. Combination Therapy

N-(2-acetyl-4,6-dimethylphenyl)-3-{[(3,4 dimethyl-5-isoxazolyl)amino]sulfonyl}-2-thiophenecarboxamide formulations provided herein can be employed alone or in combination with other suitable therapeutic agents useful in the treatment of the diseases treated by these formulations. For example, the formulations can be administered in combination with other compounds known to modulate the activity of endothelin receptor.

Further, the formulations provided herein can be employed in combination with endothelin antagonists known in the art and include, but are not limited to a fermentation product of Streptomyces misakiensis, designated BE-18257B which is a cyclic pentapeptide, cyclo(D-Glu-L-Ala-allo-D-lle-L-Leu-D-Trp); cyclic pentapeptides related to BE-18257B, such as cyclo(D-Asp-Pro-D-Val-Leu-D-Trp) (BQ-123) (see, U.S. Pat. No. 5,114,918 to Ishikawa et al.; see, also, EP A1 0 436 189 to BANYU PHARMACEUTICAL CO., LTD (Oct. 7, 1991)); and other peptide and non-peptidic ETA antagonists have been identified in, for example, U.S. Pat. Nos. 6,432,994; 6,683,103; 6,686,382; 6,248,767; 6,852,745; 5,783,705; 5,962,490; 5,594,021; 5,571,821; 5,591,761; 5,514,691; 5,352,800; 5,334,598; 5,352,659; 5,248,807; 5,240,910; 5,198.548; 5,187,195; 5,082,838; 6,953,780; 6,946,481; 6,852,745; 6,835,741; 6,673,824; 6,670,367; and 6,670,362. These include other cyclic pentapeptides, acyltripeptides, hexapeptide analogs, certain anthraquinone derivatives, indanecarboxylic acids, certain N-pyriminylbenzenesulfonamides, certain benzenesulfonamides, and certain naphthalenesulfonatnides (Nakajima et al. (1991) J. Antibiot. 44:1348-1356; Miyata et al. (1992) J. Antibiot. 45:74-8; Ishikawa et al. (1992) J. Med. Chem. 35:2139-2142; U.S. Pat. No. 5,114,918 to Ishikawa et al.; EP A1 0 569 193; EP A1 0 558 258; EP A1 0 436 189 to BANYU PHARMACEUTICAL CO., LTD (Oct. 7, 1991); Canadian Patent Application 2,067,288; Canadian Patent Application 2,071,193; U.S. Pat. No. 5,208,243; U.S. Pat. No. 5,270,313; U.S. Pat. No. 5,612,359, U.S. Pat. No. 5,514,696, U.S. Pat. No. 5,378,715; Cody et al. (1993) Med. Chem. Res. 3:154-162; Miyata et al. (1992) J. Antibiot 45:1041-1046; Miyata et al. (1992) J. Antibiot 45:1029-1040, Fujimoto et al. (1992) FEBS Lett. 305:41-44: Oshashi et al. (1002) J. Antibiot 45:1684-1685; EP A1 0 496 452; Clozel etal. (1993) Nature 365:759-761; International Patent Application WO93/08799; Nishikibe et al. (1993) Life Sci. 52:717-724; and Benigni et al. (1993) Kidney Int. 44:440-444). Numerous sulfonamides that are endothelin peptide antagonists are also described in U.S. Pat. Nos. 5,464,853; 5,594,021; 5,591,761; 5,571,821; 5,514,691; 5,464,853: International PCT application No. 96/31492; and International PCT application No. WO 94/27979.

Further endothelin antagonists described in the following documents, incorporated herein by reference in their entirety, are exemplary of those contemplated for use in combination with the formulations provided herein: U.S. Pat. No. 5,420,123; U.S. Pat. No. 5,965,732; U.S. Pat. No. 6,080,774; U.S. Pat. No. 5,780,473; U.S. Pat. No. 5,543,521; WO 96/06095; WO 95/08550; WO 95/26716; WO 96/11914; WO 95/26360; EP 601386; EP 633259; U.S. Pat. No. 5,292,740; EP 510526; EP 526708; WO 93/25580; WO 93/23404; WO 96/04905; WO 94/21259; GB 2276383; WO 95/03044; EP 617001; WO 95/03295; GB 2275926; WO 95/08989; GB 2266890; EP 496452; WO 94/21590; WO 94/21259; GB 2277446; WO 95/13262; WO 96/12706; WO 94/24084; WO 94/25013; U.S. Pat. No. 5,571,821; WO 95/04534; WO 95/04530; WO 94/02474; WO 94/14434; WO 96/07653; WO 93/08799; WO 95/05376; WO 95/12611; DE 4341663; WO 95/15963; WO 95/15944; EP 658548; EP 555537; WO 95/05374; WO 95/05372; U.S. Pat. No. 5,389,620; EP 628569; JP 6256261; WO 94/03483; EP 552417; WO 93/21219; EP 436189; WO 96/11927; JP 6122625; JP 7330622; WO 96/23773; WO 96/33170; WO 96/15109; WO 96/33190; U.S. Pat. No. 5,541,186; WO 96/19459; WO 96/19455; EP 713875; WO 95/26360; WO 96/20177; JP 7133254; WO 96/08486; WO 96/09818; WO 96/08487; WO 96/04905; EP 733626; WO 96/22978; WO 96/08483; JP 8059635; JP 7316188; WO 95/33748; WO 96/30358; U.S. Pat. No. 5,559,105; WO 95/35107; JP 7258098; U.S. Pat. No. 5,482,960; EP 682016: GB 2295616; WO 95/26957; WO 95/33752; EP 743307; and WO 96/31492; such as the following compounds described in the recited documents: BQ-123 (Ihara, M., et al., “Biological Profiles of Highly Potent Novel Endothelin Antagonists Selective for the ET_{A} Receptor”, Life Sciences, Vol. 50(4), pp. 247-255 (1992)); PD 156707 (Reynolds, E., et al., “Pharmacological Characterization of PD 156707, an Orally Active ET_{A} Receptor Antagonist”, The Journal of Pharmacology and Experimental Therapeutics, Vol. 273(3), pp. 1410-1417 (1995)); L-754,142 (Williams, D. L., et al., “Pharmacology of L-754,142, a Highly Potent, Orally Active, Nonpeptidyl Endothelin Antagonist”. The Journal of Pharmacology and Experimental Therapeutics, Vol. 275(3),. pp. 1518-1526 (1995)); SB 209670 (Ohlstein, E. H., et al., “SB 209670, a rationally designed potent nonpeptide endothelin receptor antagonist”, Proc. Natl. Acad. Sci. USA, Vol. 91. pp. 8052-8056 (1994)); SB 217242 (Ohlstein, E. H., et al., “Nonpeptide Endothelin Receptor Antagonists. VI:Pharmacological Characterization of SB 217242, A Potent and Highly Bioavailable Endothelin Receptor Antagonist”, The Journal of Pharmacology and Experimental Therapeutics, Vol. 276(2), pp. 609-615 (1996)); A-127722 (Opgenorth, T. J., et al., “Pharmacological Characterization of A-127722: An Orally Active and Highly Potent E.sub.TA-Selective Receptor Antagonist”, The Journal of Pharmacology and Experimental Therapeutics, Vol. 276(2), pp.473-481 (1996)); TAK-044 (Masuda, Y., et al., “Receptor Binding and Antagonist Properties of a Novel Endothelin Receptor Antagonist, TAK-044 {Cyclo [D-α-Aspartyl-3-[(4-Phenylpiperazin-1-yl)Carbonyl]-L-Alanyl-L-α-Aspartyl-D-2-(2-Thienyl)Glycyl-L-Leucyl-D-Tryptophyl]Disodium Salt}, in Human Endothelin_{A} and Endothelin_{B} Receptors”, The Journal of Pharmacology and Experimental Therapeutics, Vol. 279(2), pp. 675-685 (1996)); bosentan (Clozel, M., et al., “Pharmacological Characterization of Bosentan, A New Potent Orally Active Nonpeptide Endothelin Receptor Antagonist”, The Journal of Pharmacology and Experimental Therapeutics, Vol. 270(1), pp. 228-235 (1994)). In one embodiment, the compositions provided herein can be administered in combination with sitaxsentan.

The formulations provided herein can also be administered in combination with other classes of compounds. Exemplary classes of compounds for combinations herein include endothelin converting enzyme (ECE) inhibitors, such as phosphoramidon; thromboxane receptor antagonists such as ifetroban; potassium channel openers; thrombin inhibitors (e.g., hirudin and the like); growth factor inhibitors such as modulators of PDGF activity; platelet activating factor (PAF) antagonists; anti-platelet agents such as GPIIb/IIIa blockers (e.g., abdximab, eptifibatide, and tirofiban). P2Y(AC) antagonists (e.g., clopidogrel, ticlopidine and CS-747), and aspirin; anticoagulants such as warfarin, low molecular weight heparins such as enoxaparin, Factor VIIa Inhibitors, and Factor Xa Inhibitors, renin inhibitors; angiotensin converting enzyme (ACE) inhibitors such as captopril, zofenopril, fosinopril, ceranapril, alacepril, enalapril, delapril, pentopril, quinapril, ramipril, lisinopril and salts of such compounds; neutral endopeptidase (NEP) inhibitors; vasopepsidase inhibitors (dual NEP-ACE inhibitors) such as omapatrilat and gemopatrilat; HMG CoA reductase Inhibitors such as pravastatin, lovastatin, atorvastatin, simvastatin, NK-104 (a.k.a. itavastatin, or nisvastatin or nisbastatin) and ZD-4522 (also known as rosuvastatin, or atavastatin or visastatin): squalene synthetase inhibitors; fibrates; bile acid sequestrants such as questran; niacin; anti-atherosclerotic agents such as ACAT inhibitors; MTP Inhibitors: calcium channel blockers such as amlodipine besylate; potassium channel activators; alpha-adrenergic agents, beta-adrenergic agents such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide or benzothlazide as well as ethacrynic acid, tricrynafen, chlorthalidone, furosenilde, musolimine, bumetanide, triamterene, amiloride and spironolactone and salts of such compounds; thrombolytic agents such as tissue plasminogen activator (tPA), recombinant tPA, streptokinase, urokinase, prourokinase and anisoylated plasminogen streptokinase activator complex (APSAC); anti-diabetic agents such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists such as spironolactone and eplerenone; growth hormone secretagogues; aP2 inhibitors; non-steroidal antiinflammatory drugs (NSAIDS) such as aspirin and ibuprofen; phosphodiesterase inhibitors such as PDE III inhibitors (e.g., cilostazol) and PDE V inhibitors (e.g., sildenafil, vardenafil, tadalafil); protein tyrosine kinase inhibitors; antiinflammatories; antiproliferatives such as methotrexate, FK506 (tacrolimus, Prograf), mycophenolate and mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes): antimetabolites such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, octreotide acetate; microtubule-disruptor agents, such as ecteinascidins or their analogs and derivatives: microtubule-stablizing agents such as pacitaxel (Taxol®), docetaxel (Taxotere®), and epothilones A-F or their analogs or derivatives; plant-derived products, such as vinca alkaloids, epipodophyllotoxins, taxanes; and topoisomerase inhibitors: prenyl-protein transferase inhibitors: and miscellaneous agents such as, hydroxyurea, procarbazine, mitotane, hexamethylmelamine, platinum coordination complexes such as cisplatin, satraplatin, and carboplatin); cyclosporins; steroids such as prednisone or dexamethasone; gold compounds; cytotoxic drugs such as azathiprine and cyclophosphamide: TNF-alpha inhibitors such as tenidap; anti-TNF antibodies or soluble TNF receptor such as etanercept (Enbrel) rapamycin (sirolimus or Rapamune), leflunimide (Arava); and cyclooxygenase-2 (COX-2) inhibitors such as celecoxib (Celebrex) and rofecoxib (Vioxx).

I. Article of Manufacture

Also provided are articles of manufacture, containing packaging material and a formulation of N-(2-acetyl-4,6-dimethylphenyl)-3-{[(3,4 dimethyl-5-isoxazolyl)amino]sulfonyl}-2-thiophenecarboxamide provided herein within the packaging material, and a label that indicates that the formulation is used for treating an endothelin-mediated disorder.

The articles of manufacture provided herein contain packaging materials. Packaging materials for use in packaging pharmaceutical products are well known to those of skill in the art. See, e.g., U.S. Pat. Nos. 5,323,907; 5,052,558; and 5,033,352. Examples of pharmaceutical packaging materials include, but are not limited to, vials, containers, syringes, bottles, and any packaging material suitable for a selected formulation and intended mode of administration and treatment.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

### Example 1

### Preparation of N-(2-acetyl-4,6-dimethylphenyl)-3-{[(3,4 dimethyl-5-isoxazolyl)amino]sulfonyl}-2-thiophenecarboxamide.

Step 1: Preparation of Compound 1:

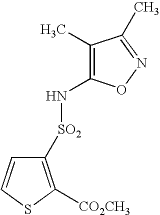

To a 250 mL round-bottom flask equipped with a magnetic stir bar was added 20.0 gm of 5-amino-3,4-dimethylisoxazole, 50 mL of pyridine, and 2.0 gm (catalytic amount) of dimethylaminopyridine. The mixture was cooled in an ice bath as 21.5 gm of 2-carboxymethyl-3-thiophenesulfonyl chloride was added in portions. The flask was sealed, the ice bath removed, and the reaction stirred at room temperature overnight. The majority of the pyridine was removed by rotary evaporation and the residual materials partitioned between ethyl acetate and 2N HCl. The layers were separated and the aqueous layer extracted with ethyl acetate (2×). The combined extracts were washed with dilute HCL (2×), brine (2×), and then dried over magnesium sulfate. Filtration and condensation by rotary evaporation yielded 23.2 gm of compound 1 as an oil.

Step 2A: Preparation of Compound 2.

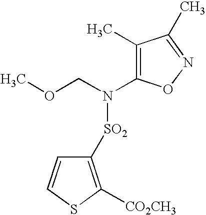

To a 1L round-bottom flask equipped with a magnetic stir bar and dropping funnel was added 23.1 gm of compound 1, 500 mL of methylene chloride, and 28.4 gm of diisopropylamine. The reaction was cooled in an ice bath and 6.0 mL of bromomethylmethyl ether was added dropwise. The ice bath was removed and the reaction stirred at room temperature overnight. At this point, 200 mL of water was added and the reaction stirred for 30 min. The layers were separated and the aqueous layer extracted (2×) with methylene chloride. The combined organic layers were then washed with 0.5 N HCl, water, saturated sodium bicarbonate, brine, and finally dried over magnesium sulfate. Filtration and rotary evaporation yielded an oil which was further purified by silica gel chromatography using 25-30% ethyl acetate/hexane as the eluant to afford 21.5 gm of compound 2 as an oil.

Step 2B: Preparation of Compound 3.

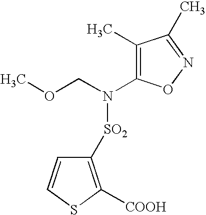

To a 500 mL round bottom flask equipped with a magnetic stir bar was added 21.4 gm of compound 2, 120 mL of tetrahydrofuran, and 120 mL of 1N sodium hydroxide. The reaction was rapidly stirred until complete reaction (approximately 3-4 hr). The majority of the tetrahydrofuran was removed by rotary evaporation and the residual materials mixed with 50 mL of water. This mixture was then acidified by the addition of 130 mL of 1N HCl and then extracted with 200 mL (2×) of ethyl acetate. The combined extracts were washed with water (50 mL), then brine (50 mL), and finally dried with magnesium sulfate. Filtration and condensation by rotary evaporation yielded 20.1 gm of compound 3 as a yellow oil which solidified upon standing.

Step 2C: Preparation of Compound 4.

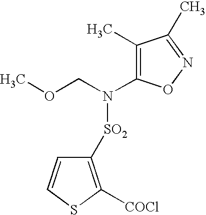

To a 1 L round bottom flask equipped with a magnetic stir bar and dropping funnel was added 19.7 gm of compound 3,200 mL of methylene chloride, and 5 drops of pyridine. A solution of 128 mL of oxalyl chloride in 100 mL of methylene chloride was added dropwise. The dropping funnel was then replaced with a reflux condenser and the reaction heated to gentle reflux for 3 hr. after which it was condensed by rotary evaporation to yield 20.9 gm of compound 4 as a brown solid. This material was used directly in Step 3 without further purification.

Step 3: Preparation of Compound 6.

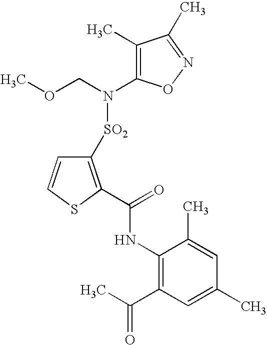

To a 1-L round bottom flask equipped with a magnetic stir bar and dropping funnel was added 18.5 gm of 2-acetyl-4,6-dimethylaniline (5) and 150 mL of methylene chloride. To this was added dropwise a solution of 20.7 gm of compound 4 dissolved in 350 mL of methylene chloride. The reaction was stirred at room temperature for 3 hr. and then condensed by rotary evaporation. To the residual materials was added 200 mL of ether and the mixture was filtered. The filter cake was washed with 3×100 mL of ether. The combined filtrates were washed with 3×φmL of 1N HCl followed by 100 mL each with water, sat. sodium bicarbonate, and brine. The solution was then dried with magnesium sulfate, filtered, and condensed by rotary evaporation to yield a semi-crystalline material. This material was triturated with 200 mL of ether to yield 23.7 gm of compound 6 as a white solid.

Step 4A: Preparation of N-(2-acetyl-4,6-dimethylphenyl)-3-{[(3,4 dimethyl-5-isoxazolyl)amino]sulfonyl}-2-thiophenecarboxamide

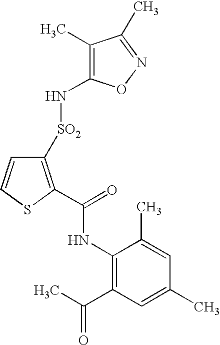

To a 500 mL round-bottom flask equipped with a magnetic stir bar and reflux condenser was added 23.7 gm of compound 6. 180 mL of methanol, and 90 mL of conc. HCl. The mixture was heated to reflux for 4 hr. Heating was discontinued and the mixture stirred and cooled with an ice bath. After approximately 30 min. the mixture was filtered and the filter cake washed with a mixture of water and methanol to yield 18.3 gm of N-(2-acetyl-4,6-dimethylphenyl)-3-{[(3,4 dimethyl-5-isoxazolyl)amino]sulfonyl}-2-thiophenecarboxamide. This material was recrystallized from ethyl acetate/hexane to give 16.8 gm of material as a white solid: mp 158-160° C.

### Example 2

### Preparation of N-(2-acetyl-4,6-dimethylphenyl)-3-{[(3,4 dimethyl-5-isoxazolyl)amino]sulfonyl}-2-thiophenecarboxamide.

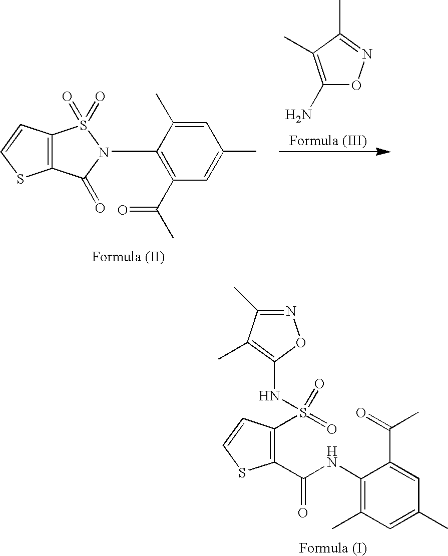

At 0° C.: To 1.6 g 60% NaH in mineral oil was added a solution of 1.12 g of the compound of Formula (III) in 10 ml DMF. After stirring for 15 minutes a solution of 3.35 g of the compound of Formula (II in 10 ml DMF was added drop wise. The reaction was stirred for 2 hours followed by a slow addition of 50 ml 2 N HCl (caution, excess NaH). The resulting suspension was extracted with toluene (4×25 ml). The organic layers were combined and washed with 2 N HCl (4×25 ml) followed by extraction with sat. NaHCO₃ (4×10 ml). The bicarbonate layers were combined and acidified with conc. HCl to pH˜1-2 and extracted with EtOAc (3×25 ml). The EtOAc layers were combined and washed with 2 N HCl (25 ml), 2 N HCl/brine (25 ml), dried over MgSO₄ and concentrated in vacuo to yield 4.0 g N-(2-acetyl-4,6-dimethylphenyl)-3-{[(3,4 dimethyl-5-isoxazolyl)amino]sulfonyl}-2-thiophenecarboxamideas a tan solid with a purity >97%. Crystallization of 3.2 g crude N-(2-acetyl-4,6-dimethylphenyl)-3-{[(3,4 dimethyl-5-isoxazolyl)amino]sulfonyl}-2-thiophenecarboxamide from hot EtOH gave 2.75 g of the title compound as an off white solid in >99% purity by HPLC. ¹H NMR (500 MHz, DMSO-d₆): δ 1.65 (s, 3H), 2.08 (s, 3H), 2.22 (s, 3H), 2.32 (s, 3H), 2.47 (s, 3H), 7.27 (brs, 1H), 7.33 (d, J=5.2 Hz, 1H), 7.38 (brs, 1H),. 7.86 (d, J=5.2 Hz. 1H) and 10.26 (brs. 1H). ¹³C. NMR (125 MHz, DMSO-d₆): δ 5.8, 10.3,17.7, 20.4, 29.1, 105.7. 126.5. 127.9 128.7, 129.8, 133.9, 135.8, 136.2, 136.5, 138.3, 139.9, 155.2, 159.1, 161.4 and 200.5 ppm. MS (ESI) m/z: 446.08 [M−H]⁻.

### Example 2a

### Preparation of the Compound of Formula (II)

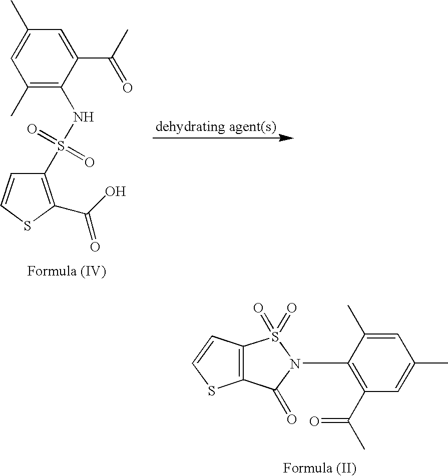

To a solution of 5.7 g of the compound of Formula (IV) in 60 ml EtOAc was added 3.1 g CDI. After stirring for 4 hours the reaction mixture was concentrated in vacuo. Crystallization from hot EtOAc gave 4.25 g of the compound of Formula (II) as a white solid in >99% by HPLC. ¹H NMR (400 MHz, CDCl₃): δ 2.40 (s, 3H), 2.43 (s, 3H), 2.53 (s, 3H), 7.35 (brs, 1H), 7.41 (d, J=5.1 Hz, 1H), 7.45 (brs, 1H) and 7.91 (d, J=5.1 Hz, 1H) ppm. MS (ESI) m/z: 693.08 [2M+H]⁺, and 336.00 [M+H]⁺.

### Example 2b

### Preparation of the Compound of Formula (IV)

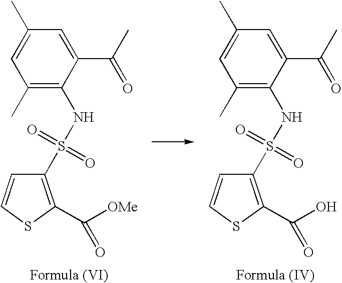

To a solution of 6.25 g of the compound of Formula (VI) in 60 ml THF was added 30 ml 2N NaOH. After overnight stirring the reaction mixture was quenched with 10 ml conc. HCl followed by extraction with EtOAc (2×). The organic layers were combined and washed with 2 N HCl (2×), 2N HCl/brine (1×), dried over MgSO₄ and concentrated in vacuo to yield 6.0 g of the compound of Formula (IV) as a light green sticky foam. Tituration with DCM gave the product as a dry white solid in >99% purity by HPLC. ¹H NMR (400 MHz, CDCl₃): δ 2.14 (s, 3H), 2.28 (s. 3H), 2.31 (s, 3H), 7.17 (d, J=5.1 Hz, 1H), 7.27 (brs, 1H), 7.30 (brs, 1H), 7.64 (d, J=5.1 Hz, 1H) and 8.91 (brs, 1) ppm. MS (ESI) m/z: 720.08 [2M+H]⁺, 376.04 [M+Na]⁺ and 354.06 [M+H]⁺.

### Example 2c

### Preparation of the Compound of Formula (VI)

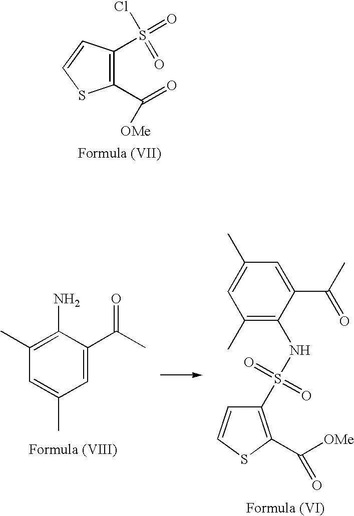

To a solution of 4.4 g of the compound of Formula (VIII) in 20 ml pyridine was added 6.0 g the compound of Formula (VII). The reaction mixture was stirred overnight followed by addition of 50 ml 6 N HCl. The resulting suspension was extracted with DCM (2×). The organic layers were combined and washed with 2 N HCl (2×), 2 N HCl/brine, dried over MgSO₄ and concentrated in vacuo. Crystallization from hot EtOAc/hexanes (1:1, 20 ml) gave 6.33 g of the compound of Formula (VI) as an off-white solid in >99% purity by HPLC. ¹H NMR (400 MHz, CDCl₃): δ 2.14 (s, 3H), 2.32 (s, 3H), 2.42 (s, 3H), 4.04 (s, 3H), 7.18 (brs, 1H), 7.22 (brs, 1H), 7.28 (d, J=5.1 Hz, 1H), 7.40 (d, J=5.1 Hz, 1H) and 9.16 (brs, 1H) ppm. MS (ESI) m/z: 757.12 [2M+H]⁺ and 368.04 [M+H]⁺.

### Exemplary Formulations of N-(2-acetyl-4,6-dimethylphenyl)-3-{[(3,4 dimethyl-5-isoxazolyl)amino]sulfonyl}-2-thiophenecarboxamide:

The following examples provide exemplary IV and tablet formulations of N-(2-acetyl-4,6-dimethylphenyl)-3-{[(3,4 dimethyl-5-isoxazolyl)amino]sulfonyl}-2-thiophenecarboxamide and their stability studies.

A. IV Formulations

### Example 3

A Prototype Stability Formulation of N-(2-acetyl-4,6-dimethylphenyl)-3-{[(3,4 dimethyl-5-isoxazolyl)amino]sulfonyl}-2-thiophenecarboxamide

A prototype stability study was designed to probe the sensitivity of 5 formulations to pH, buffer concentration and drug concentration. The formulations listed below were examined for pH (7, 8 and 11), buffer concentration (20 mM versus 50 mM at pH 8) and drug concentration (0.5 mg/mL versus 50 mg/mL)
Formula A: 50 mg/ml, 50 mM buffer, pH 8.0
Formula B: 50 mg/ml, 20 mM buffer, pH 8.0
Formula C: 50 mg/ml, 50 mM buffer, pH 7.0
Formula D: 50 mg/ml, 50 mM buffer, pH˜11.0
Formula E: 0.5 mg/ml, 20 mM buffer, pH 8.0

Each prototype formulation was compounded at a scale of 250 mL. The target final pH values were achieved within a range of ±0.3 pH units. The osmolality of the formulations was determined in-process and all were hypotonic ranging from 74 mOsm/kg for E to 254 mOsm/kg for D. NaCl was added to formulation B to raise the tonicity slightly. These formulations were placed on stability at 5° C., 25° C. and 40° C. for time points consisting of initial, 2 weeks, 1 month, 3 months and 6 months. Based on the stability data summarized in Tables IV-VIII, formulation B was selected for further studies.

TABLE IV
Formulation: E, N-(2-acetyl-4,6-dimethylphenyl)-3-{[(3,4 dimethyl-5-isoxazolyl)amino] sulfonyl}-
2-thiophenecarboxamide at 0.5 mg/mL in 20 mM phosphate (pH 8)

| | 25° C./65% RH Storage | | | | 40° C./75% RH Storage | | | |
|---|---|---|---|---|---|---|---|---|
| | | Total Rel. | | | | Total Rel. | | |
| Time | Assay | Substances | | | Assay | Substances | | |
| Points | (% LC) | (%) | pH | Appearance | (% LC) | (%) | pH | Appearance |
| Initial | 98.2 | <0.05 | 7.68 | * | 98.2 | <0.05 | 7.68 | * |
| 0.5 Mon | 100.1 | <0.05 | 7.68 | NC | 99.5 | <0.05 | 7.72 | NC |
| 1 Mon | 100.1 | <0.05 | 7.70 | NC | 100.5 | <0.05 | 7.72 | NC |

* Clear, colorless solution, free of visible particulates
NC = No change

TABLE V
Formulation: B, N-(2-acetyl-4,6-dimethylphenyl)-3-{[(3,4 dimethyl-5-isoxazolyl)amino] sulfonyl}-
2-thiophenecarboxamide at 50 mg/mL in 20 mM phosphate (pH 8)

| | 25° C./65% RH Storage | | | | 40° C./75% RH Storage | | | |
|---|---|---|---|---|---|---|---|---|
| | | Total Rel. | | | | Total Rel. | | |
| Time | Assay | Substances | | | Assay | Substances | | |
| Points | (% LC) | (%) | pH | Appearance | (% LC) | (%) | pH | Appearance |
| Initial | 98.6 | <0.05 | 8.17 | * | 98.6 | <0.05 | 8.17 | * |
| 0.5 Mon | 100.2 | <0.05 | 8.19 | NC | 99.9 | <0.05 | 8.20 | NC |
| 1 Mon | 100.6 | <0.05 | 8.20 | NC | 101.2 | 0.07 | 8.17 | NC |

* Clear, yellow solution, free of visible particulates
NC = No change

TABLE VI
Formulation: C, N-(2-acetyl-4,6-dimethylphenyl)-3-{[(3,4 dimethyl-5-isoxazolyl)amino] sulfonyl}-
2-thiophenecarboxamide at 50 mg/mL in 50 mM phosphate (pH 7)

| | 25° C./65% RH Storage | | | | 40° C./75% RH Storage | | | |
|---|---|---|---|---|---|---|---|---|
| | | Total Rel. | | | | Total Rel. | | |
| Time | Assay | Substances | | | Assay | Substances | | |
| Points | (% LC) | (%) | pH | Appearance | (% LC) | (%) | pH | Appearance |
| Initial | 99.4 | <0.05 | 6.94 | * | 99.4 | <0.05 | 6.94 | * |
| 0.5 Mon | 100.5 | <0.05 | 6.93 | NC | 100.4 | <0.05 | 6.92 | NC |
| 1 Mon | 101.4 | 0.15 | 7.00 | NC | 100.6 | 0.24 | 6.96 | NC |

* Clear, yellow solution, free of visible particulates
NC = No change

TABLE VII
Formulation: A, N-(2-acetyl-4,6-dimethylphenyl)-3-{[(3,4 dimethyl-5-isoxazolyl)amino] sulfonyl}-
2-thiophenecarboxamide at 50 mg/mL in 50 mM phosphate (pH 8)

| | 25° C./65% RH Storage | | | | 40° C./75% RH Storage | | | |
|---|---|---|---|---|---|---|---|---|
| | | Total Rel. | | | | Total Rel. | | |
| Time | Assay | Substances | | | Assay | Substances | | |
| Points | (% LC) | (%) | pH | Appearance | (% LC) | (%) | pH | Appearance |
| Initial | 99.0 | <0.05 | 7.87 | * | 99.0 | <0.05 | 7.87 | * |
| 0.5 Mon | 100.8 | <0.05 | 7.89 | NC | 100.6 | <0.05 | 7.87 | NC |
| 1 Mon | 101.4 | <0.05 | 7.92 | NC | 102.0 | 0.06 | 7.93 | NC |

* Clear, yellow solution, free of visible particulates
NC = No change

TABLE VIII
Formulation: D, N-(2-acetyl-4,6-dimethylphenyl)-3-{[(3,4 dimethyl-5-isoxazolyl)amino] sulfonyl}-
2- thiophenecarboxamide at 50 mg/mL in 50 mM phosphate (pH 11)

| | 25° C./65% RH Storage | | | | 40° C./75% RH Storage | | | |
|---|---|---|---|---|---|---|---|---|
| | | Total Rel. | | | | Total Rel. | | |
| Time | Assay | Substances | | | Assay | Substances | | |
| Points | (% LC) | (%) | pH | Appearance | (% LC) | (%) | pH | Appearance |
| Initial | 98.9 | <0.05 | 11.11 | * | 98.9 | <0.05 | 11.11 | * |
| 0.5 Mon | 100.6 | 0.08 | 11.16 | NC | 99.8 | 0.63 | 11.13 | NC |
| 1 Mon | 100.8 | 0.16 | 11.20 | NC | 100.4 | 1.01 | 11.12 | NC |

* Clear, yellow solution, free of visible particulates
NC = No change

### Example 4

Compatibility Studies with 50 mg/mL N-(2-acetyl-4,6-dimethylphenyl)-3-{[(3,4 dimethyl-5-isoxazolyl)amino]sulfonyl}-2-thiophenecarboxamide in 20 mM phosphate buffer with NaCl

The formulation of 50 mg/mL N-(2-acetyl-4,6-dimethylphenyl)-3-{[(3,4 dimethyl-5-isoxazolyl)amino]sulfonyl}-2-thiophenecarboxamide in 20 mM phosphate buffer (pH 8.0) with NaCl was manufactured on a 500 mL scale and tested for filter, tubing, terminal sterilization feasibility, stopper compatibility studies and freeze/ thaw studies. Filter compatibility was based on the use of a Millipore durapore 0.2um membrane syringe filter testing prefiltration, 1st sample from the filter and post filtration sample. Masterflex size 15 platinum cure tubing was exposed to light and ambient temperature for a period of 24 hours for tubing compatibility. Samples were exposed to one and two standard liquid autoclave cycles of 121° C. over a period of 20 minutes for terminal sterilization feasibility studies. Stopper compatibility studies test samples at room temperature and 40° C. conditions having samples inverted and upright over a period of 7 days. Data showed that the formulation was compatible with filter, tubing, and stopper. There was a decrease in purity for terminal sterilized for one and two cycles of 121° C. over a period of 20 minutes (Tables IX-XII).

TABLE IX
Compatibility Studies (Filter) for 50 mg/mL N-(2-acetyl-4,6-
dimethylphenyl)-3-{[(3,4 dimethyl-5-isoxazolyl)amino]
sulfonyl}-2-thiophenecarboxamide in 20 mM phosphate (pH 8) with NaCl

| | Assay | Total Rel. | | |
|---|---|---|---|---|
| Timepoints | (% LC) | Substances (%) | pH | Appearance |
| Prefiltration | 97.1 | <0.05 | 8.28 | * |
| 1^{st} sample | 97.0 | <0.05 | 8.11 | NC |
| from filter | | | | |
| Postfiltration | 96.8 | <0.05 | 8.31 | NC |
| (Control) | | | | |

* Clear, yellow solution, free of visible particulates,
NC = No change

TABLE X
Compatibility Studies (Tubing) for 50 mg/mL N-(2-acetyl-4,6-
dimethylphenyl)-3-{[(3,4 dimethyl-5-isoxazolyl)amino]
sulfonyl}-2-thiophenecarboxamide in 20 mM phosphate (pH 8) with NaCl

| | Assay | Total Rel. | | |
|---|---|---|---|---|
| Timepoints | (% LC) | Substances (%) | pH | Appearance |
| Control | 97.3 | <0.05 | 8.30 | * |
| Exposed to | 98.8 | <0.05 | 8.14 | NC |
| Tubing | | | | |

* Clear, yellow solution, free of visible particulates,
NC = No change

TABLE XI
Compatibility Studies (Terminal Sterilization) for 50 mg/mL
N-(2-acetyl-4,6-dimethylphenyl)-3-{[(3,4 dimethyl-5-
isoxazolyl)amino] sulfonyl}-2-thiophenecarboxamide
in 20 mM phosphate (pH 8) with NaCl

| | | Total Rel. | | |
|---|---|---|---|---|
| | | Sub- | | |
| | Assay | stances | | Appear- |
| Timepoints | (% LC) | (%) | pH | ance |
| Control (1) | 97.2 | <0.05 | 8.33 | * |
| Control (2) | 96.5 | <0.05 | 8.34 | NC |
| Exposed to 1 Autoclave Cycle (1) | 96.6 | 0.20 | 8.27 | NC |
| Exposed to 1 Autoclave Cycle (2) | 96.9 | 0.19 | 8.28 | NC |
| Exposed to 2 Autoclave Cycle (1) | 96.4 | 0.30 | 8.27 | NC |
| Exposed to 2 Autoclave Cycle (2) | 96.6 | 0.35 | 8.25 | NC |

* Clear, yellow solution, free of visible particulates,
NC = No change

TABLE XII
Compatibility Studies (Stopper) for 50 mg/mL N-(2-acetyl-4,6-dimethylphenyl)-3-{[(3,4dimethyl-5-
isoxazolyl)amino]sulfonyl}-2-thiophenecarboxamide in 20 mM phosphate (pH 8) with NaCl

| | Room Temperature | | | | 40° C. | | | |
|---|---|---|---|---|---|---|---|---|
| | | Total Rel. | | | | Total Rel. | | |
| Time | Assay | Substances | | | Assay | Substances | | |
| Points | (% LC) | (%) | pH | Appearance | (% LC) | (%) | pH | Appearance |
| T = 0 | 97.1 | <0.05 | 8.27 | * | — | — | — | — |
| Day 1 (upright) | 96.9 | <0.05 | 8.30 | NC | 97.7 | <0.05 | 8.32 | * |
| Day 2 (upright) | 96.2 | <0.05 | 8.31 | NC | 96.6 | <0.05 | 8.31 | NC |
| Day 5 (upright) | 96.4 | <0.05 | 8.30 | NC | 97.8 | <0.05 | 8.32 | NC |
| Day 7 (upright) | 94.2 | <0.05 | 8.29 | NC | 97.4 | <0.05 | 8.32 | NC |
| Day 1 (inverted) | 97.3 | <0.05 | 8.32 | * | 96.7 | <0.05 | 8.24 | * |
| Day 2 (inverted) | 96.6 | <0.05 | 8.35 | NC | 96.9 | <0.05 | 8.31 | NC |
| Day 5 (inverted) | 97.1 | <0.05 | 8.30 | NC | 97.9 | <0.05 | 8.31 | NC |
| Day 7 (inverted) | 96.7 | <0.05 | 8.29 | NC | 97.3 | <0.05 | 8.31 | NC |

* Clear, yellow solution, free of visible particulates,
NC = No change

Stability Study - Effect of pH and Buffer Agents

| | | | Assay (%) | | Total Related (%) | | |
|---|---|---|---|---|---|---|---|
| Batch | Timepoint | Condition | Rep 1 | Rep 2 | Rep 1 | Rep 2 | Note |
| #1 | ATST | ATST | 100.2 | 99.4 | 0.04 | 0.04 | |
| pH 6 Acetate | 2 W | 40 C./75% RH | 99.5 | 100.0 | <0.04 | <0.04 | |
| 1 mg/mL | | 60 C. | 100.5 | 99.9 | 0.13 | 0.12 | |
| | 1 M | 40 C./75% | 100.3 | 100.4 | 0.07 | 0.07 | |
| | | RH | | | | | |
| | | 60 C. | 100.8 | 100.3 | 0.20 | 0.21 | |
| | 3 M | 40 C./75% | 99.4 | 99.1 | 0.12 | 0.12 | |
| | | RH | | | | | |
| | | 60 C. | 99.1 | 98.3 | 0.56 | 0.59 | |
| #2 | ATST | ATST | 98.2 | 99.0 | 0.04 | <0.04 | |
| pH 6 | 2 W | 40 C./75% | 98.8 | 99.3 | 0.05 | 0.06 | |
| Phosphate | | RH | | | | | |
| 1 mg/mL | | 60 C. | 98.7 | 99.5 | 0.17 | 0.18 | |
| | 1 M | 40 C./75% | 99.7 | 99.4 | 0.09 | 0.09 | |
| | | RH | | | | | |
| | | 60 C. | 98.2 | 98.0 | 0.33 | 0.33 | |
| | 3 M | 40 C./75% | 98.1 | 98.0 | 0.17 | 0.18 | |
| | | RH | | | | | |
| | | 60 C. | 97.2 | 97.5 | 0.92 | 0.93 | |
| #3 | ATST | ATST | 107.2 | 107.5 | <0.04 | <0.04 | |
| pH 6 Citrate | 2 W | 40 C./75% RH | 106.9 | 108.3 | <0.04 | 0.06 | |
| 1 mg/mL | | 60 C. | 107.5 | 108.0 | 0.14 | 0.14 | |
| | 1 M | 40 C./75% | 107.7 | 107.9 | 0.07 | 0.07 | |
| | | RH | | | | | |
| | | 60 C. | 107.4 | 107.3 | 0.23 | 0.23 | |
| | 3 M | 40 C./75% | 106.8 | 106.7 | 0.13 | 0.14 | |
| | | RH | | | | | |
| | | 60 C. | 106.4 | 106.4 | 0.65 | 0.65 | |
| #4 | ATST | ATST | 100.1 | 101.1 | <0.04 | <0.04 | |
| pH 7 | 2 W | 40 C./75% | 101.0 | 99.6 | 0.14 | 0.16 | |
| Phosphate | | RH | | | | | |
| 50 mg/mL | | 60 C. | 99.8 | 100.7 | 0.44 | 0.36 | |
| | 1 M | 40 C./75% | 101.4 | 100.7 | 0.23 | 0.25 | |
| | | RH | | | | | |
| | | 60 C. | 99.5 | 99.4 | 0.73 | 0.70 | |
| | 3 M | 40 C./75% | 99.1 | 99.8 | 0.56 | 0.58 | |
| | | RH | | | | | |
| | | 60 C. | 99.2 | 97.7 | 1.34 | 1.33 | |
| #5 | ATST | ATST | 100.3 | 100.9 | <0.04 | <0.04 | pH 7.8 |
| pH 8 | 2 W | 40 C./75% | 100.5 | 100.8 | 0.05 | <0.04 | |
| Phosphate | | RH | | | | | |
| 50 mg/mL | | 60 C. | 99.7 | 100.7 | 0.19 | 0.19 | |
| | 1 M | 40 C./75% RH | 101.4 | 100.1 | 0.08 | 0.08 | |
| | | 60 C. | 99.6 | 100.6 | 0.37 | 0.35 | |
| | 3 M | 40 C./75% | 99.8 | 100.6 | 0.17 | 0.18 | |
| | | RH | | | | | |
| | | 60 C. | 98.9 | 99.0 | 0.78 | 0.77 | pH 7.6 |
| #6 | ATST | ATST | 101.2 | 101.4 | <0.04 | <0.04 | |
| pH 9 | 2 W | 40 C./75% | 98.6 | 99.7 | <0.04 | <0.04 | |
| Phosphate | | RH | | | | | |
| 50 mg/mL | | 60 C. | 101.1 | 101.0 | 0.17 | 0.17 | |
| | 1 M | 40 C./75% | 99.1 | 100.5 | 0.08 | 0.07 | |
| | | RH | | | | | |
| | | 60 C. | 101.7 | 101.2 | 0.30 | 0.31 | |
| | 3 M | 40 C./75% | 99.1 | 100.2 | 0.12 | 0.13 | |
| | | RH | | | | | |
| | | 60 C. | 99.1 | 99.8 | 0.67 | 0.68 | |
| #7 | ATST | ATST | 100.3 | 100.2 | <0.04 | <0.04 | pH 8.0 |
| pH 8 TRIS | 2 W | 40 C./75% | 99.8 | 100.1 | 0.05 | <0.04 | |
| 50 mg/mL | | RH | | | | | |
| | | 60 C. | 100.6 | 98.8 | 0.16 | 0.16 | |
| | 1 M | 40 C./75% RH | 100.6 | 100.8 | 0.06 | 0.06 | |
| | | 60 C. | 99.7 | 100.0 | 0.32 | 0.31 | |
| | 3 M | 40 C./75% | 99.2 | 100.1 | 0.11 | 0.11 | |
| | | RH | | | | | |
| | | 60 C. | 99.0 | 98.8 | 0.72 | 0.71 | pH 8.2 |
| #8 | ATST | ATST | 100.1 | 100.1 | <0.04 | <0.04 | pH 8.2 |
| pH 8 | 2 W | 40 C./75% | 100.1 | 100.4 | 0.05 | 0.04 | |
| Triethanolamine | | RH | | | | | |
| 50 mg/mL | | 60 C. | 100.0 | 99.5 | 0.13 | 0.13 | |
| | 1 M | 40 C./75% | 100.8 | 101.2 | 0.06 | 0.06 | |
| | | RH | | | | | |
| | | 60 C. | 99.5 | 100.0 | 0.25 | 0.25 | |
| | 3 M | 40 C./75% RH | 99.5 | 100.6 | 0.10 | 0.10 | w. |
| | | | | | | | other |
| | | | | | | | related |
| | | 60 C. | 99.5 | 99.1 | 0.81 | 0.82 | pH 8.2 |

### Example 5

### Development of a Single Solution Compounding Procedure

Single solution compounding procedures were developed for N-(2-acetyl-4,6-dimethylphenyl)-3-{[(3,4 dimethyl-5-isoxazolyl)amino]sulfonyl}-2-thiophene-carboxamide formulations containing 50 mg/ml drug in 20 and 50 mM phosphate buffer solution at either pH 7 or 8 and the development data is summarized in Table XIII

A drug/NaOH solution was prepared with 1.1 equivalents of NaOH. After the drug was fully solubilized, the required amount of sodium phosphate dibasic heptahydrate was added to the batch solution. In the next step, the required amount of sodium phosphate monobasic monohydrate was added to the batch. The total amount of phosphate buffer salt added was calculated to result in specific final buffer concentrations. The ratio of the two buffer salts was varied to determine the proper amounts to achieve the desired final pH values.

TABLE XIII
Development of Single Solution Compounding Procedures for N-(2-
acetyl-4,6-dimethylphenyl)-3-{[(3,4 dimethyl-5-
isoxazolyl)amino] sulfonyl}-2-thiophenecarboxamide
(20 mL Scale)

| | |
|---|---|
| | Resultant |
| Process | pH |

50 mM pH 7.0 ± 0.3 Formulation

| | |
|---|---|
| N-(2-acetyl-4,6-dimethylphenyl)-3-{[(3,4 dimethyl-5- | 11.49 |
| isoxazolyl)amino] sulfonyl}-2-thiophenecarboxamide | |
| dissolved at 50 mg/mL in 1.1 eq. solution of NaOH | |
| Add 0.14 g of dibasic phosphate to 20 mL of drug/NaOH | 10.73 |
| solution | |
| Add 0.066 g of monobasic phosphate to intermediate solution | 6.95 |

50 mM pH 8 ± 0.3 Formulation

| | |
|---|---|
| N-(2-acetyl-4,6-dimethylphenyl)-3-{[(3,4 dimethyl-5- | 11.48 |
| isoxazolyl)amino] sulfonyl}-2-thiophenecarboxamide | |
| dissolved at 50 mg/mL in 1.1 eq. solution of NaOH | |
| Add 0.225 g of dibasic phosphate to 20 mL of drug/NaOH | 10.49 |
| solution | |
| Add 0.022 g of monobasic phosphate to intermediate solution | 7.91 |

20 mM pH 7 ± 0.3 Formulation

| | |
|---|---|
| N-(2-acetyl-4,6-dimethylphenyl)-3-{[(3,4 dimethyl-5- | 11.52 |
| isoxazolyl)amino] sulfonyl}-2-thiophenecarboxamide | |
| dissolved at 50 mg/mL in 1.1 eq. solution of NaOH | |
| Add 0.05 g of dibasic phosphate to 20 mL of drug/NaOH | 11.11 |
| solution | |
| Add 0.029 g of monobasic phosphate to intermediate solution | 7.23 |

20 mM pH 8 ± 0.5 Formulation

| | |
|---|---|
| N-(2-acetyl-4,6-dimethylphenyl)-3-{[(3,4 dimethyl-5- | 10.74 |
| isoxazolyl)amino] sulfonyl}-2-thiophenecarboxamide | |
| dissolved dissolved at 50 mg/mL in 1.1 eq. solution of NaOH | |
| Add 0.09 g of dibasic phosphate to 20 mL of drug/NaOH | 10.31 |
| solution | |
| Add 0.009 g of monobasic phosphate to intermediate solution | 8.51 |

### Example 6

### Preparation of 50 mg/mL N-(2-acetyl-4,6-dimethylphenyl)-3-{[(3,4 dimethyl-5-isoxazolyl)amino]sulfonyl}-2-thiophenecarboxamide in 20 mM phosphate buffer (pH 8.0±1.0) with NaCl

A single solution compounding procedure described in Example 4 was used to prepare a 30L batch of 50 mg/mL N-(2-acetyl-4,6-dimethylphenyl)-3-{[(3,4 dimethyl-5-isoxazolyl)amino]sulfonyl}-2-thiophenccaboxamide in 20 mM phosphate buffer (pH 8.0±1.0) with NaCl. Compounding additions were added as described above. A higher pH resulted after the addition on sodium phosphate monobasic. The formulation was pH adjusted to target with 0.1N HCl.

### Example 7

### Manufacture of a 55 litre batch of N-(2-acetyl-4,6-dimethylphenyl)-3-{[(3,4 dimethyl-5-isoxazolyl)amino]sulfonyl}-2-thiophenecarboxamide sterile solution for injection (50 mg/ml)

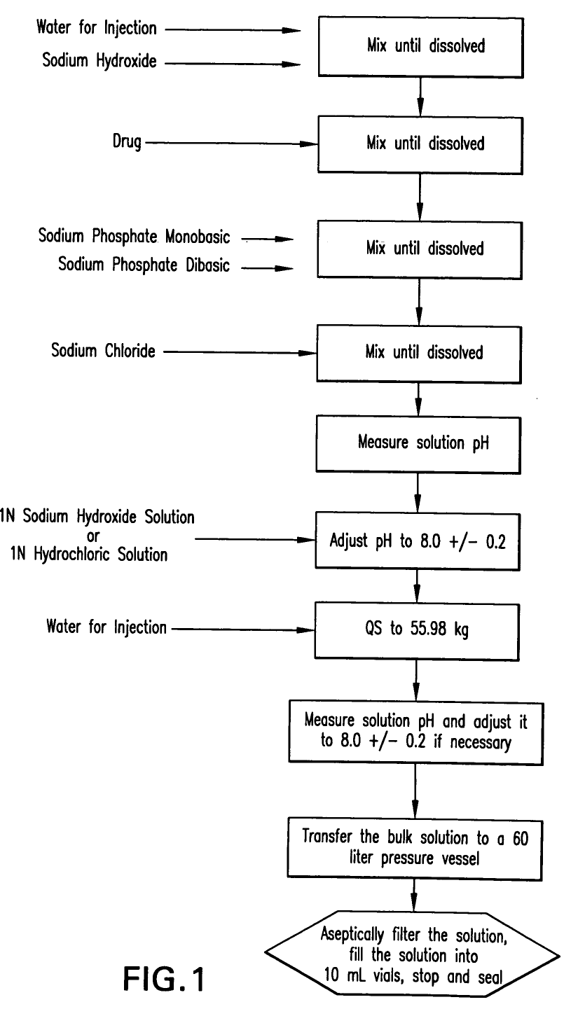

A flow diagram for the manufacture of a 55 litre batch of N-(2-acetyl-4,6-dimethylphenyl)-3-{[(3,4 dimethyl-5-isoxazolyl)amino]sulfonyl}-2-thiophenecarboxamide sterile solution for injection (50 mg/ml) is provided in FIG. 1. and a stepwise description of the manufacturing process is as follows:

### Stepwise Description of the Manufacturing Process

Step 1: Preparation of 1.0 Normal Solution of Sodium Hydroxide
a. Water for injection was charged to a 1 Litre Pyrex volumetric flask.
b. Sodium Hydroxide pellets were charged to the water and the stoppered flask was gently swirled until a clear solution was obtained.
c. Additional amount of Water for injection was added to the above solution to a final volume of 1.0 Litre.

Step 2: Preparation of 1.0 Normal Solution of Hydrochloric Acid
a. Water for injection was charged to a 1 Litre Pyrex volumetric flask.
b. Hydrochloric Acid was charged to the water and the stoppered flask was gently swirled until a uniform solution was obtained.
c. Additional Water for injection was added to the above solution to a final volume of 1.0 Litre.
   Step 3: N-(2-acetyl-4,6-dimethylphenyl)-3-{[(3,4 dimethyl-5-isoxazolyl)amino]sulfonyl}-2-thiophenecarboxamide Solution
a. Water for injection was charged to a SS stock pot.
b. Sodium Hydroxide pellets were added and mixture stirred until complete dissolution.
c. N-(2-acetyl-4,6-dimethylphenyl)-3-{[(3,4 dimethyl-5-isoxazolyl)amino]sulfonyl}-2-thiophenecarboxamide drug substance was added and stirred until a clear solution was obtained.
d. To this were added sodium phosphate dibasic, heptahydrate and stirred until a clear solution was obtained.
e. Sodium phosphate monobasic monohydrate was added and stirred until a clear solution was obtained.
f. Sodium chloride was added and stirred until a clear solution was obtained.
g. The pH of the clear solution was measured and, if necessary, adjusted to pH 8.0±0.2 by the addition of either 1.0 Normal sodium hydroxide or 1.0 Normal hydrochloric acid.
h. Water for injection was charged to bring the solution to its final required weight.
i. The pH of the final solution was measured and, if necessary, adjusted to pH 8.0±0.2 by the addition of either 1.0 Normal sodium hydroxide or 1.0 Normal hydrochloric acid.
j. The final bulk solution was transferred to a 60 Litre pressure vessel.

Step 4: Sterile Filtration and Vial Filling
a. The final bulk solution was aseptically filtered with a Shibuya filter and filling machine into 10 mL clear glass vials which were also stoppered and sealed.

B. Oral Tablet Formulations:

### Example 8

### Manufacturing Process for N-(2-acetyl-4,6-dimethylphenyl)-3-{[(3,4 dimethyl-5-isoxazolyl)amino]sulfonyl}-2-thiophenecarboxamide 1.0 mg Tablets

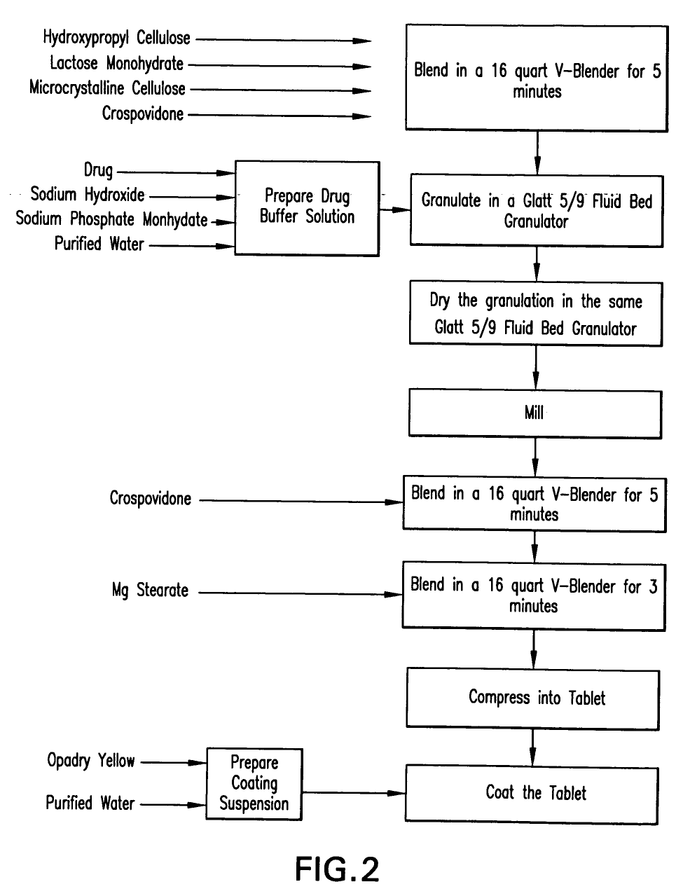

For 1.0 mg tablet, the drug was dissolved in a buffer solution and sprayed as granulating agent onto the intragranular materials during the fluid bed granulation process to ensure the drug content uniformity. A representative manufacturing flow diagram for the 1.0 mg coated tablets is shown in FIG. 2.

The batch formula used to manufacture a 4 kg batch of 1.0 mg coated tablets is summarized in Table XIV.

TABLE XIV
Batch Formula for a 4-kg Tablet Batch of N-(2-acetyl-4,6-
dimethylphenyl)-3-{[(3,4 dimethyl-5-isoxazolyl)amino] sulfonyl}-
2-thiophenecarboxamide 1.0 mg Coated Tablets

| | Amount per Batch |
|---|---|
| Component | (grams) |
| Intragranular Components | |
| Hydroxypropyl Cellulose (Klucel EXF) | 160.0 |
| Lactose Monohydrate 310 | 2,755.0 |
| Microcrystalline Cellulose (Avicel PH101) | 800.0 |
| Crospovidone CL (Kollidone CL) | 40.0 |
| Total | 3,755.0 |
| Granulating Agents | |
| N-(2-acetyl-4,6-dimethylphenyl)-3-{[(3,4 | 40.0¹ |
| dimethyl-5-isoxazolyl)amino] sulfonyl}-2- | |
| thiophenecarboxamide | |
| Sodium Hydroxide | 4.0¹ |
| Sodium Phosphate Monobasic, Granular | 1.2 |
| AR | |
| Purified Water | (1,400.0)² |
| Total | 46.2 |
| Extragranular Components | |
| Crospovidone CL (Kollidone CL) | 160.0 |
| Magnesium Stearate (Non-Bovine #5712) | 40.0 |
| Total | 200.0 |
| Film Coating Suspension | |
| Opadry Yellow 03F92230 | 120.0 |
| Purified Water | (1,080)² |
| Total | 120.0 |

¹The amount of sodium and sodium phosphate monobasic may be varied to obtain a granulation solution of pH 7.5-8.5.
²Water is removed during processing

The process steps are summarized below:
1. Preparation of N-(2-acetyl-4,6-dimethylphenyl)-3-{[(3,4 dimethyl-5-isoxazolyl)amino]sulfonyl}-2-thiophenecarboxamide Drug Granulation Solution:
a. A 1% sodium hydroxide solution was prepared by adding sodium hydroxide to purified water and mixing until a clear solution was obtained;
b. Milled N-(2-acetyl-4,6-dimethylphenyl)-3-{[(3,4 dimethyl-5-isoxazolyl)amino]sulfonyl}-2-thiophenecarboxamide was added while mixing to the 1% sodium hydroxide solution. Mixing was continued until a clear solution was produced;
c. A sodium phosphate monobasic solution was prepared by adding sodium phosphate monobasic to purified water and mixing until a clear solution was obtained;
d. The sodium phosphate monobasic was added solution to the N-(2-acetyl-4,6-dimethylphenyl)-3-{[(3,4 dimethyl-5-isoxazolyl)amino]sulfonyl}-2-thiophenecarboxamidesolution while mixing. The pH was adjusted with sodium phosphate monobasic or sodium hydroxide solution if necessary to pH 7.5-8.5.
   2. Granulating and Drying
a. Screened hydroxypropyl cellulose, lactose monohydrate, microcrystalline cellulose, and crospovidone were added into a 16 quart V-blender and blended for five minutes;
b. The blended material was charged into a Glatt 5/9 fluid bed granulator;
c. The N-(2-acetyl-4,6-dimethylphenyl)-3-{[(3,4 dimethyl-5-isoxazolyl)amino]sulfonyl}-2-thiophenecarboxamidebuffed granulation solution was added to the blended material in the fluid bed granulator using appropriate granulating parameters;
d. After completion of the granulation process, the granulated material was dried until a moisture content of less than 2.5% was achieved;
   3. Milling and Final Blending
e. The dried granulation was passed through a Comil;
f. The milled granulation and the screened crospovidone were added to a 16 quart V-blender and blended for five minutes;
g. The screened magnesium stearate was added to the V-blend and blended for another three minutes.
   4. Tableting
h. The final blend was compressed into 100 mg tablets using ¼″ round standard concave tooling with target tablet hardness of 4.5 kp.
   5. Coating
i. Opadry Yellow was added into purified water and the suspension was mixed for a minimum of one hour;
j. The N-(2-acetyl-4,6-dimethylphenyl)-3-{[(3,4 dimethyl-5-isoxazolyl)amino]sulfonyl}-2-thiophenecarboxamide 1.0 mg uncoated tablets were coated in a Compulab 24 fitted with a 19″ pan using the requisite amount of coating suspension to obtain 3% coating.

### Example 9

### Manufacturing Process for N-(2-acetyl-4,6-dimethylphenyl)-3-{[(3,4 dimethyl-5-isoxazolyl)amino]sulfonyl}-2-thiophenecarboxamide 10 and 50 mg Tablets

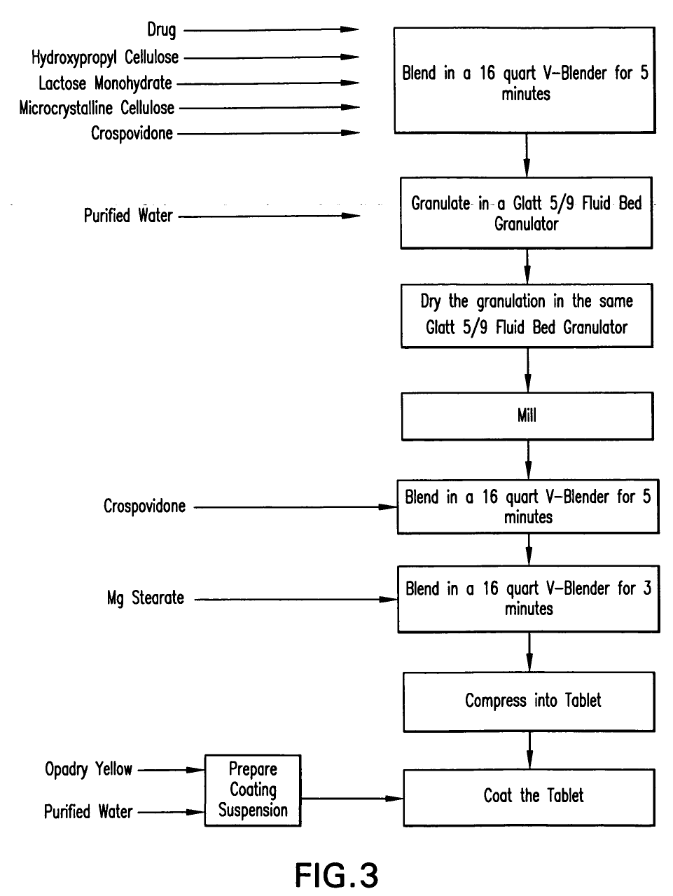

For the 10 mg and 50 mg tablets, N-(2-acetyl-4,6-dimethylphenyl)-3-{[(3,4 dimethyl-5-isoxazolyl)amino]sulfonyl}-2-thiophenecarboxamide was blended with the intragranular materials in the fluid blend granulator and water was used as the granulating agent. A representative manufacturing flow diagram for the 10 mg and 50 mg coated tablets is shown in FIG. 3.

The batch formula used to manufacture a 4 kg batch of 10 and 50 mg coated tablets is summarized in Tables XV and XVI.

TABLE XV
Batch Formula for a 4-kg Tablet Batch of N-(2-acetyl-4,6-
dimethylphenyl)-3-{[(3,4 dimethyl-5-isoxazolyl)amino] sulfonyl}-2-
thiophenecarboxamide10 mg Coated Tablets

| | Amount per Batch |
|---|---|
| Component | (grams) |
| Intragranular Components | |
| Milled N-(2-acetyl-4,6-dimethylphenyl)-3- | 400.0 |
| {[(3,4 dimethyl-5-isoxazolyl)amino] | |
| sulfonyl}-2-thiophenecarboxamide | |
| Hydroxypropyl Cellulose (Klucel EXF) | 160.0 |
| Lactose Monohydrate 310 | 2,400.0 |
| Microcrystalline Cellulose (Avicel PH101) | 800.0 |
| Crospovidone CL (Kollidone CL) | 40.0 |
| Total | 3,800.0 |
| Granulating Agents | |
| Purified Water | (1,400.0)¹ |
| Total | 0.0 |
| Extragranular Components | |
| Crospovidone CL (Kollidone CL) | 160.0 |
| Magnesium Stearate (Non-Bovine #5712) | 40.0 |
| Total | 200.0 |
| Film Coating Suspension | |
| Opadry Yellow 03F92230 | 120.0 |
| Purified Water | (1,080)¹ |
| Total | 120.0 |

¹Water is removed during processing

TABLE XVI
Batch Formula for a 4-kg Tablet Batch of N-(2-acetyl-4,6-
dimethylphenyl)-3-{[(3,4 dimethyl-5-isoxazolyl)amino] sulfonyl}-
2-thiophenecarboxamide50 mg Coated Tablets

| | Amount per Batch |
|---|---|
| Component | (grams) |
| Intragranular Components | |
| Milled N-(2-acetyl-4,6-dimethylphenyl)-3- | 2000.0 |
| {[(3,4 dimethyl-5-isoxazolyl)amino] | |
| sulfonyl}-2-thiophenecarboxamide | |
| Hydroxypropyl Cellulose (Klucel EXF) | 160.0 |
| Lactose Monohydrate 310 | 800.0 |
| Microcrystalline Cellulose (Avicel PH101) | 800.0 |
| Crospovidone CL (Kollidone CL) | 40.0 |
| Total | 3,800.0 |
| Granulating Agents | |
| Purified Water | (1,400.0)¹ |
| Total | 0.0 |
| Extragranular Components | |
| Crospovidone CL (Kollidone CL) | 160.0 |
| Magnesium Stearate (Non-Bovine #5712) | 40.0 |
| Total | 200.0 |
| Film Coating Suspension | |
| Opadry Yellow 03F92230 | 120.0 |
| Purified Water | (1,080)¹ |
| Total | 120.0 |

¹Water is removed during processing

The process steps are summarized below:
1 Granulating and Drying
   a. Screened N-(2-acetyl-4,6-dimethylphenyl)-3-{[(3,4 dimethyl-5-isoxazolyl)amino]sulfonyl}-2-thiophenecarboxamide milled drug substance, hydroxypropyl cellulose, lactose monohydrate, microcrystalline cellulose, and crospovidone were added into a 16 quart V-blender and blended for five minutes;
   b. The blended material was charged into a Glatt 5/9 fluid bed granulator;
   k. Purified water was added to the blended material in the fluid bed granulator using appropriate granulating parameters:
   l. After completion of the granulation process, the granulated material was dried until a moisture content of less than 2.5% was achieved;
2. Milling and Final Blending
   a. The dried granulation was passed through a Comil;
   b. The milled granulation, the screened crospovidone was added to a 16 quart V-blender and blended for five minutes,
   c. The screened magnesium stearate was added to the V-blend and blended for another three minutes.
3. Tableting
   a. The final blend was compressed into 100 mg tablets using ¼″ round standard concave tooling with target tablet hardness of 4.5 kp.
4. Coating
   m. Opadry Yellow was added into purified water and the suspension was mixed for a minimum of one hour;
   n. The N-(2-acetyl-4,6-dimethylphenyl)-3-{[(3,4 dimethyl-5-isoxazolyl)amino]sulfonyl}-2-thiophenecarboxamide 1.0 mg uncoated tablets were coated in a Compulab 24 fitted with a 19″ pan using the requisite amount of coating suspension to obtain 3% coating.

All of the references cited herein are incorporated by reference in their entirety. While the invention has been described with respect to the particular embodiments, it will be apparent to those skilled in the art that various changes and modifications may be made without departing from the spirit and scope of the invention as recited by the appended claims.

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. An intravenous formulation comprising N-(2-acetyl-4,6-dimethylphenyl)-3-{[(3,4 dimethyl-5-isoxazolyl)amino]sulfonyl}-2-thiophenecarboxamide and a buffer.

2. The intravenous formulation of claim 1, wherein N-(2-acetyl-4,6-dimethylphenyl)-3-{[(3,4 dimethyl-5-isoxazolyl)amino]sulfonyl}-2-thiophenecarboxamide is present in a concentration from about 30 mg/mL to about 60 mg/mL.

3. The intravenous formulation of claim 2, wherein the concentration of N-(2-acetyl-4,6-dimethylphenyl)-3-{[(3,4 dimethyl-5-isoxazolyl)amino]sulfonyl}-2-thiophenecarboxamide is about 50 mg/mL.

4. The intravenous formulation of claim 1, wherein the buffer is a phosphate or citrate buffer.

5. The intravenous formulation of claim 4, wherein the buffer is a phosphate buffer.

6. The intravenous formulation of claim 5, wherein the phosphate buffer is present in a concentration of about 20 mM to about 50 mM.

7. The intravenous formulation of claim 6, wherein the concentration of the phosphate buffer is about 20 mM.

8. The intravenous formulation of claim 1, wherein the formulation has a pH of about 7-9.

9. The intravenous formulation of claim 1, wherein the formulation has a pH of about 7, 8 or 9.

10. The intravenous formulation of claim 6, wherein the phosphate buffer concentration is about 20 mM and the pH is about 8.

11. The intravenous formulation of claim 3, wherein the concentration of N-(2-acetyl-4,6-dimethylphenyl)-3-{[(3,4 dimethyl-5-isoxazolyl)amino]sulfonyl}-2-thiophenecarboxamide is about 50 mg/mL, the phosphate buffer concentration is about 20 mM and the pH is about 8.

12. An oral tablet formulation comprising N-(2-acetyl-4,6-dimethylphenyl)-3-{[(3,4 dimethyl-5-isoxazolyl)amino]sulfonyl}-2-thiophenecarboxamide, hydroxypropyl cellulose (Klucel EXF), lactose monohydrate 310, microcrystalline cellulose. cospovidone CL, sodium hydroxide, sodium phosphate monobasic and magnesium stearate.

13. The oral tablet formulation of claim 12, wherein the N-(2-acetyl-4,6-dimethylphenyl)-3-{[(3,4 dimethyl-5-isoxazolyl)amino]sulfonyl}-2-thiophenecarboxamide is present in an amount ranging from about 1% to about 50% of the total weight of the tablet.

14. The oral tablet formulation of claim 13, wherein the amount of N-(2-acetyl-4,6-dimethylphenyl)-3-{[(3,4 dimethyl-5-isoxazolyl)amino]sulfonyl}-2-thiophenecarboxamide is about 1% of the total weight of the tablet.

15. The oral tablet formulation of claim 13, wherein the amount of N-(2-acetyl-4,6-dimethylphenyl)-3-{[(3,4 dimethyl-5-isoxazolyl)amino]sulfonyl}-2-thiophenecarboxamide is about 10% of the total weight of the tablet.

16. The oral tablet formulation of claim 13, wherein the amount of N-(2-acetyl-4,6-dimethylphenyl)-3-{[(3,4 dimethyl-5-isoxazolyl)amino]sulfonyl}-2-thiophenecarboxamide is about 50% of the total weight of the tablet.

17. The oral tablet formulation of claim 13, wherein the amount of N-(2-acetyl-4,6-dimethylphenyl)-3-{[(3,4 dimethyl-5-isoxazolyl)amino]sulfonyl}-2-thiophenecarboxamide is about 1 mg.

18. The oral tablet formulation of claim 13, wherein the amount of N-(2-acetyl-4,6-dimethylphenyl)-3-{[(3,4 dimethyl-5-isoxazolyl)amino]sulfonyl}-2-thiophenecarboxamide is about 10 mg.

19. The oral tablet formulation of claim 13, wherein the amount of N-(2-acetyl-4,6-dimethylphenyl)-3-{[(3,4 dimethyl-5-isoxazolyl)amino]sulfonyl}-2-thiophenecarboxamide is about 50 mg.

20. The oral tablet formulation of claim 12, wherein lactose monohydrate 310 is present in an amount from about 20% to 80% of the total weight of the tablet.

21. The oral tablet formulation of claim 20, wherein the amount of lactose monohydrate 310 is about 20%, 60% or 69% of the total w eight of the tablet.

22. The oral tablet formulation of claim 12, wherein microcrystalline cellulose is present in an amount from about 5% to 40% of the total weight of the tablet.

23. The oral tablet formulation of claim 22, wherein the amount of microcrystalline cellulose is about 20% of the total weight of the tablet.

24. The oral tablet formulation of claim 12, wherein hydroxypropyl methylcellulose is present in an amount from about 1% to about 5% of the total weight of the tablet.

25. The oral tablet formulation of claim 24, wherein the amount of hydroxypropyl methylcellulose is about 4% of the total weight of the tablet.

26. The oral tablet formulation of claim 12, wherein sodium hydroxide is present in an amount from about 0.01% to about 1% of the total weight of the tablet.

27. The oral tablet formulation of claim 26, wherein the amount of sodium hydroxide is about 0.1% of the total weight of the tablet.

28. The oral tablet formulation of claim 12, wherein sodium phosphate monobasic is present in an amount from about 0.01% to about 1% of the total weight of the tablet.

29. The oral tablet formulation of claim 28, wherein sodium phosphate monobasic is about 0.03% of the total weight of the tablet.

30. The oral tablet formulation of claim 12, wherein crospovidone CL is present in an amount from about 0.5% to about 5% of the total weight of the tablet.

31. The oral tablet formulation of claim 30, wherein the amount of crospovidone CL is about 1% or about 4% of the total weight of the tablet.

32. The oral tablet formulation of claim 12, wherein magnesium stearate is present in an amount from about 0.1% to about 5% of the total weight of the tablet.

33. The oral tablet formulation of claim 32, wherein the amount of magnesium stearate is about 1% or about 4% of the total weight of the tablet.

34. The oral tablet formulation of claim 12 further comprising a coating.

35. The oral tablet formulation of claim 12, wherein the coating comprises opadry yellow.

36. The oral tablet formulation of claim 35, wherein the opadry yellow is present in an amount at about 3% of the total weight of the tablet.

37. The oral tablet formulation of claim 12, wherein the tablet comprises about 4.0% hydroxypropyl cellulose (Klucel EXF), about 68.87% lactose monohydrate 310, about 20% microcrystalline cellulose, about 1% cospovidone CL, about 1% N-(2-acetyl-4,6-dimethylphenyl)-3-{[(3,4 dimethyl-5-isoxazolyl)amino]sulfonyl}-2-thiophenecarboxamide, about 0.1% sodium hydroxide, about 0.1% sodium phosphate monobasic, about 4% magnesium stearate and about 3% opadry yellow.

38. The oral tablet formulation of claim 12, wherein the tablet comprises about 4.0 mg hydroxypropyl cellulose (Klucel EXF), about 68.87 mg lactose monohydrate 310, about 20 mg microcrystalline cellulose, about 1 mg cospovidone CL, about 1 mg N-(2-acetyl-4,6-dimethylphenyl)-3-{[(3,4 dimethyl-5-isoxazolyl)amino]sulfonyl}-2-thiophenecarboxamide, about 0.1 mg sodium hydroxide, about 0.1 mg sodium phosphate monobasic, about 4 mg magnesium stearate and about 3 mg opadry yellow.

39. The oral tablet formulation of claim 12, wherein the tablet comprises about 10% N-(2-acetyl-4,6-dimethylphenyl)-3-{[(3,4 dimethyl-5-isoxazolyl)amino]sulfonyl}-2-thiophenecarboxamide, about 4.0% hydroxypropyl cellulose (Klucel EXF), about 60% lactose monohydrate 310, about 20% microcrystalline cellulose, about 1% cospovidone CL, about 4% magnesium stearate and about 3% opadry yellow.

40. The oral tablet formulation of claim 12, wherein the tablet comprises about 10 mg N-(2-acetyl-4,6-dimethylphenyl)-3-{[(3,4 dimethyl-5-isoxazolyl)amino]sulfonyl}-2-thiophenecarboxamide, about 4 mg hydroxypropyl cellulose (Klucel EXF), about 60 mg lactose monohydrate 310, about 20 mg microcrystalline cellulose, about 1 mg cospovidone CL, about 4 mg magnesium stearate and about 3 mg opadry yellow.

41. The oral tablet formulation of claim 12, wherein the tablet comprises about 50% N-(2-acetyl-4,6-dimethylphenyl)-3-{[(3,4 dimethyl-5-isoxazolyl)amino]sulfonyl}-2-thiophenecarboxamide, about 4% hydroxypropyl cellulose (Klucel EXF), about 20% lactose monohydrate 310, about 20% microcrystalline cellulose about 1% cospovidone CL, about 4% magnesium stearate and about 3% opadry yellow.

42. The oral tablet formulation of claim 12, wherein the tablet comprises about 50 mg N-(2-acetyl-4,6-dimethylphenyl)-3-{[(3,4 dimethyl-5-isoxazolyl)amino]sulfonyl}-2-thiophenecarboxamide, about 4 mg hydroxypropyl cellulose (Klucel EXF), about 20 mg lactose monohydrate 310, about 20 mg microcrystalline cellulose, about 1 mg cospovidone CL, about 4 mg magnesium stearate and about 3 mg opadry yellow.

43. A combination, comprising the formulation of claim I and a sterile vessel containing a single dosage or multiple dosage amount thereof.

44. The combination of claim 43, wherein the vessel is an ampoule, vial or syringe.

45. A method for treating an endothelin-mediated disease, comprising administering an effective amount of the formulation of claim 1.

46. The method of claim 45, wherein the disease is selected from the group consisting of hypertension, cardiovascular disease, asthma, pulmonary hypertension, inflammatory diseases, ophthalmologic disease, menstrual disorders, obstetric conditions, wounds, gastroenteric disease, renal failure, immunosuppressant-mediated renal vasoconstriction, erythropoietin-mediated vasoconstriction endotoxin shock, anaphylactic shock and hemorrhagic shock.

47. An article of manufacture comprising packaging material and a formulation of claim 1, contained within the packaging material, wherein the packaging material includes a label that indicates that the formulation is used for treating an endothelin mediated disorder.

48. A method for treating an endothelin-mediated disease, comprising administering an effective amount of the formulation of claim 12.

49. The method of claim 48, wherein the disease is selected from the group consisting of hypertension, cardiovascular disease, asthma, pulmonary hypertension, inflammatory diseases, ophthalmologic disease, menstrual disorders, obstetric conditions, wounds, gastroenteric disease, renal failure, immunosuppressant-mediated renal vasoconstriction, erythropoietin-mediated vasoconstriction endotoxin shock, anaphylactic shock and hemorrhagic shock.

50. An article of manufacture comprising packaging material and a formulation of claim 12, contained within the packaging material, wherein the packaging material includes a label that indicates that the formulation is used for treating an endothelin mediated disorder.
